(19) United States
(12) Patent Application Publication
(10) Pub. No.: US 2021/0299094 A1
(43) Pub. Date: Sep. 30, 2021

(54) METHODS OF UPREGULATING TIPARP AS ANTICANCER STRATEGIES

(71) Applicant: CORNELL UNIVERSITY, Ithaca, NY (US)

(72) Inventors: Hening LIN, Ithaca, NY (US); Lu ZHANG, Ithaca, NY (US)

(21) Appl. No.: 17/260,124

(22) PCT Filed: Jul. 24, 2019
(86) PCT No.: PCT/US2019/043206

§ 371 Date: Jan. 13, 2021

Related U.S. Application Data
(60) Provisional application No. 62/702,634, filed on Jul. 24, 2018.

(51) Int. Cl. A61K 31/404; A61K 31/138; A61K 31/352; A61K 31/357; A61P 35/00; A61K 31/05

(52) U.S. Cl. CPC A61K 31/404; A61K 31/138; A61K 31/05; A61K 31/357; A61P 35/00; A61K 31/352

(57) ABSTRACT

The present disclosure is directed to a method of treating cancer in a subject in need thereof, the method comprising administering to the subject a therapeutically effective amount of a TiPARP agonist, wherein the TiPARP agonist may be, for example, a tamoxifen compound (e.g., tamoxifen or derivative thereof), flavone or derivative thereof, isoflavone or derivative thereof, diindolylmethane compound, or chlorinated dibenzo-p-dioxin (CDBD) compound or derivative thereof. The cancer may be associated with elevated expression of HIF-1α and may be selected from, for example, breast cancer, colon cancer, lung cancer, skin cancer, brain cancer, blood cancer, cervical cancer, liver cancer, prostate carcinoma, pancreas carcinoma, gastric carcinoma, ovarian carcinoma, renal cell carcinoma, mesothelioma, and melanoma. The cancer may, in some embodiments, exclude breast cancer.

### CROSS REFERENCE TO RELATED APPLICATION

This application claims the benefit of priority from U.S. Provisional Application No. 62/702,634, filed Jul. 24, 2018, the entire contents of which are incorporated herein by reference.

### STATEMENT REGARDING FEDERALLY SPONSORED RESEARCH OR DEVELOPMENT

This invention was made with government support under Grant Nos. GM086703 and OD018516, awarded by the National Institutes of Health. The government has certain rights in the invention.

### INCORPORATION BY REFERENCE OF SEQUENCE LISTING

The Sequence Listing in an ASCII text file, named as 36450PCT_8342_02_PC_SequenceListing.txt of 8 KB, created on Jul. 24, 2019, and submitted to the United States Patent and Trademark Office via EFS-Web, is incorporated herein by reference.

### BACKGROUND

ADP-ribosylation is a reversible protein post-translational modification (PTM) that transfers a single, or multiple ADP-ribosyl groups to substrate proteins (Gibson, B. A. & Kraus, W. L., Nat Rev Mol Cell Biol 13, 411-424, (2012)). Intracellular ADP-ribosylation is catalyzed by the ADP-ribosyltransferase diphtheria toxin-like (ARTDs), commonly known as poly-ADP-ribose polymerases (PARPs) (Hottiger, M. O., et al. Trends Biochem Sci 35, 208-219, (2010)). Compared to poly-ADP-ribosylation, the function of intracellular mono-ADP-ribosylation is less understood. Nevertheless, it is becoming evident that mono-ADP-ribosylation modulates important signaling pathways and it has been linked to numerous diseases, including inflammation, diabetes, neurodegeneration, and cancer (Corda, D. & Di Girolamo, M. EMBO J 22, 1953-1958, (2003); Butepage, M., et al., Cells 4, 569-595, (2015); Corda, D. & Di Girolamo, M., Sci STKE 2002, pe53, (2002); Del Vecchio, M. & Balducci, E. Mol Cell Biochem 310, 77-83, (2008); Scarpa, E. S., Fabrizio, G. & Di Girolamo, M. FEBS J 280, 3551-3562, (2013).). Tetrachlorodibenzo-p-dioxin (TCDD)-inducible poly (ADP-ribose) polymerase (TiPARP, also known as PARP7 or ARTD14) is a mono-ADP-ribosyltransferase (MacPherson, L. et al. Nucleic Acids lies 41, 1604-1621, (2013)) and TiPARP was first identified as a target gene of and hydrocarbon receptor (AHR) in response to the dioxin TCDD (Ma, Q. Arch Biochem Biophys 404, 309-316 (2002); Ma, Q. et al, Biochem Biophys Res Commun 289, 499-506, (2001)). Once expressed, TiPARP regulates transcriptional activity of AHR and liver X receptor via ADP-ribosylation (MacPherson, L. et al. Nucleic Acids Res 41, 1604-1621, (2013); Bindesboll, C. et al. Biochem J 413, 899-910, (2016)). However, the detailed function of TiPARP and its role in modulating transcription was not well understood. The transcriptional activity of both AHR and HIF-1α require their bindings with the co-activator HIF-1β (also known as and hydrocarbon receptor nuclear translocator, ARNT), as well as the recognition of GCGTG core sequence on target genes (Semenza, G. L. & Wang, G. L. Mol Cell Biol 12, 5447-5454 (1992); Jiang, B. H. et al., J Biol Chem 271, 17771-17778 (1996); Wang, G. L. et al., Proc Natl Acad Sci USA 92, 5510-5514 (1995)). Structurally, they both contain the basic-helix-loop-helix (bHLH)-PAS motif that is essential for their heterodimerization with HIF-1β (Jiang, B. H. et al., J Biol Chem 271, 17771-17778 (1996); Wang, G. L. et al., Proc Natl Acad Sci USA 92, 5510-5514 (1995)). The similarity between AHR and HIF-1α prompted us to investigate the connection between HIF-1 and TiPARP. Activated HIF-1 is a key regulator of oxygen homeostasis that mediates adaptive responses to changes in oxygenation through transcriptional activation of genes involved in glucose metabolism and cell survival (Gordan, J. D. & Simon, M. C. Curr Op in Genet Dev 17, 71-77, (2007); Semenza, G. L. Nat Rev Cancer 3, 721-732, (2003)). Due to intratumoral hypoxia and genetic mutations, HIF-1α is stabilized or overexpressed in human cancers and is often associated with increased mortality in cancer patients (Semenza, G. L. Nat Rev Cancer 3, 721-732, (2003); Semenza, G. L. Genes Dev 14, 1983-1991 (2000); Masoud, G. N. & Li, W. Acta Pharm Sin B 5, 378-389, (2015)).

### SUMMARY OF THE DISCLOSURE

The present disclosure is directed to methods for treating cancer in a subject in need thereof, the method comprising administering to the subject a therapeutically effective amount of a TiPARP agonist. In particular embodiments, the TiPARP agonist interacts with TiPARP directly. In some embodiments, the TIPARP agonist may be an agent that leads to elevated expression of the TiPARP protein. In other embodiments, the TIPARP agonist is an expression vector encoding an exogenous TiPARP protein, or more particularly, wherein the expression of the exogenous TiPARP is inducible. The TiPARP agonist may be, for example, a tamoxifen compound (e.g., tamoxifen or derivative thereof), flavone or derivative thereof, isoflavone or derivative thereof, diindolylmethane compound, or chlorinated dibenzo-p-dioxin (CDBD) compound or derivative thereof. The cancer being treated may be associated with an elevated expression of HIF-1a. The cancer may be selected from, for example, breast cancer, colon cancer, lung cancer, skin cancer, brain cancer, blood cancer, cervical cancer, liver cancer, prostate carcinoma, pancreas carcinoma, gastric carcinoma, ovarian carcinoma, renal cell carcinoma, mesothelioma, and melanoma. The cancer may, in some embodiments, be other than breast cancer, such as lung or colon cancer.

In some embodiments, the disclosure is directed to a method of treating cancer in a subject in need thereof, the method comprising administering to the subject a therapeutically effective amount of a TiPARP agonist.

In some embodiments, the TiPARP agonist is an aryl hydrocarbon receptor (AHR) agonist. In some embodiments, the TiPARP agonist is an estrogen receptor (ER) agonist.

In some embodiments, the TiPARP agonist interacts with TiPARP directly.

In some embodiments, the TiPARP agonist is a tamoxifen compound within the following generic formula:

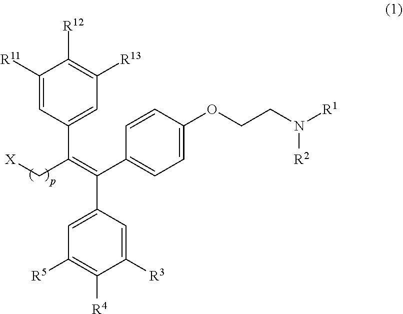

wherein:
R¹ and R² are independently selected from alkyl groups containing one to three carbon atoms, or alternatively, R¹ and R² may interconnect to form a five-membered or six-membered heterocycloalkyl ring;
R³, R⁴, and R are independently selected from hydrogen atom, halogen atom, methyl, ethyl, hydroxy (OH), methoxy (—OCH₃), and ethoxy (—OCH₂CH₃);
R¹¹, R¹² and R¹³ are independently selected from hydrogen atom, hydroxy, and methoxy groups;
X is a hydrogen atom or halogen atom; and
p is 2 or 3.

In some embodiments, the tamoxifen compound has the following structure:

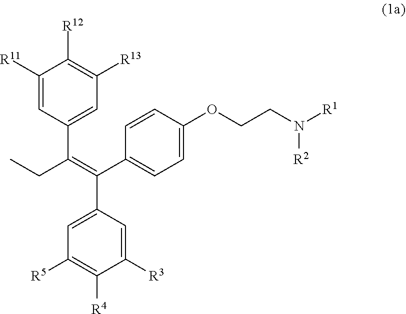

In some embodiments, R¹ and R² are independently selected from alkyl groups containing one to three carbon atoms.

In some embodiments, R¹ and R² are methyl groups, which corresponds to the following structure:

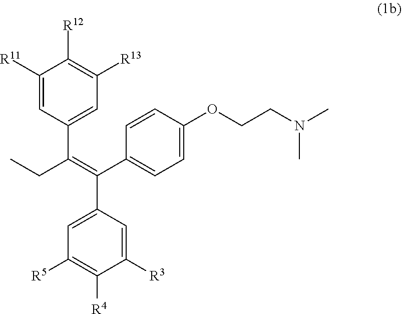

In some embodiments, R³ and R⁵ are hydrogen atoms and R⁴ is selected from the group consisting of halogen atom, methyl, ethyl, hydroxy (OH), methoxy (—OCH₃), and ethoxy (—OCH₂CH₃).

In some embodiments, R³, R⁴, and R⁵ are hydrogen atoms, which corresponds to the following structure:

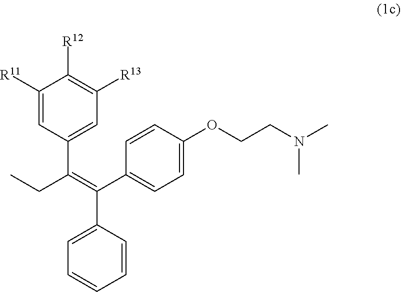

In some embodiments, at least one of R¹¹, R¹², and R¹³ is a hydroxy or methoxy group.

In some embodiments, R¹¹ and R¹³ are hydrogen atoms and R¹² is a hydroxy or methoxy group.

In some embodiments, the compound has the following structure:

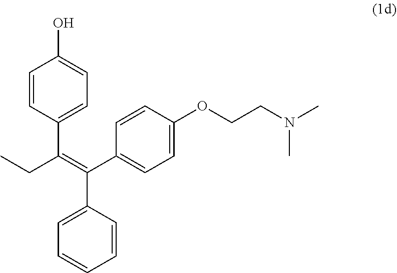 [SMILES: CC/C(=C(\c1ccccc1)c1ccc(OCCN(C)C)cc1)c1ccc(O)cc1]

In some embodiments, the TiPARP agonist is a flavone or isoflavone compound within the following generic formula:

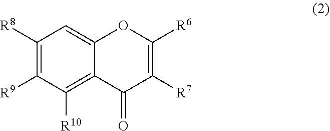

wherein:
R⁶ and R⁷ are independently selected from (i) hydrogen atom and (ii) phenyl ring optionally substituted with one or two OH and/or OCH₃ groups, provided that one of R⁶ and R⁷ is (ii);
R⁸, R⁹, and R¹⁰ are independently selected from hydrogen atom, methyl, phenyl, hydroxy, and methoxy groups, wherein said phenyl is optionally substituted with a hydroxy or methoxy group;
wherein R⁸ and R⁹ may optionally interconnect as a benzene ring, or R⁹ and R¹⁰ may optionally interconnect as a benzene ring.

In some embodiments, at least one of R⁸, R⁹, and R¹⁰ is a hydroxy group and none of R⁸, R⁹, and R¹⁰ interconnect.

In some embodiments, at least two of R⁸, R⁹, and R¹⁰ are hydroxy groups.

In some embodiments, one of R⁶ and R⁷ is a phenyl ring substituted with an OH or OCH₃ group.

In some embodiments, the TiPARP agonist has the following structure:

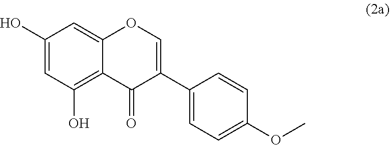 [SMILES: COc1ccc(-c2coc3cc(O)cc(O)c3c2=O)cc1]

In some embodiments, R⁹ and R¹⁰ interconnect as a benzene ring and R⁸ is a hydrogen atom, which corresponds to the following structure:

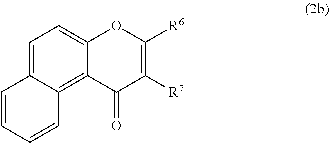

In some embodiments, one of R⁶ and R⁷ is an unsubstituted phenyl ring.

In some embodiments, the compound has the following structure:

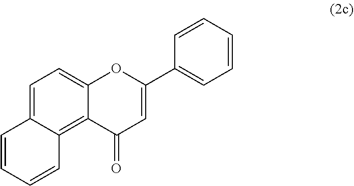 [SMILES: O=c1cc(-c2ccccc2)oc2ccc3ccccc3c12]

In some embodiments, the TiPARP agonist is a diindolylmethane compound having the following structure:

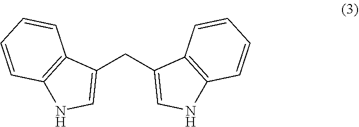 [SMILES: c1ccc2c(Cc3c[nH]c4ccccc34)c[nH]c2c1]

In some embodiments, the TiPARP agonist is a chlorinated dibenzo-p-dioxin (CDBD) compound within the following generic formula:

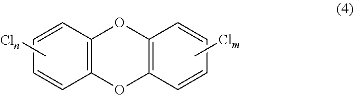

wherein n represents a number between 0 and 4, and wherein m represents a number between 0 and 4, provided that the sum of m and n is at least 1.

In some embodiments, the CDBD compound is tetrachlorodibenzo-p-dioxin (TCDD), which corresponds to the following structure:

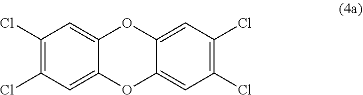 [SMILES: Clc1cc2c(cc1Cl)Oc1cc(Cl)c(Cl)cc1O2]

In some embodiments, the TIPARP agonist is an agent that leads to elevated expression of the TiPARP protein.

In some embodiments, the TIPARP agonist is an expression vector encoding an exogenous TiPARP protein.

In some embodiments, the expression of the exogenous TiPARP is inducible.

In some embodiments, the cancer is associated with elevated expression of HIF-1a.

In some embodiments, the cancer is selected from the group consisting of breast cancer, colon cancer, lung cancer, skin cancer, brain cancer, blood cancer, cervical cancer, liver cancer, prostate carcinoma, pancreas carcinoma, gastric carcinoma, ovarian carcinoma, renal cell carcinoma, mesothelioma, and melanoma.

In some embodiments, the cancer is not breast cancer.

In some embodiments, the cancer is lung or colon cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1F. TiPARP is a direct target gene of HIFs. (A) A schematic representation of TiPARP promoter. Core sequence of hypoxia-response element (HRE) is highlighted in red. (B) Expression of TiPARP mRNA in HCT116 and MCF-7 cells analyzed by qRT-PCR under normoxia/hypoxia (mean and s.d. of three independent experiments). (C) RT-PCR analysis of TiPARP mRNA level in HEK 293T treated with hypoxia-mimetic agent DMOG and DFO, or transfected with HA tagged HIF-1α. Expression of endogenous or HA-HIF-1α was detected by HIF-1α antibody on western blot (bottom), (D) Schematic representation of the luciferase reporter construct with wild type (WT) or mutated (Mut) HRE. (E) HIF-1α transactivation measured by luciferase reporter with WT or mutant HRE from TiPARP promoter. Transfection efficiencies were normalized to co-transfected Renilla-luciferase. Mean and standard deviation (s.d.) correspond to four technical replicates, representative of three independent experiments. NS, not significant. (F) ChIP assay assessing the chromatin binding of HIF-1α to HRE in TiPARP promoter, RNA polymerase II (Pol II) was used as a positive control.

FIGS. 2A-2D. TiPARP represses HIF-1 transcriptional activity. (A) HIF-1α reporter activity measured in HEK 293T cells transfected with HA-HIF-1α and HRE-luciferase, in the presence of vector, Flag-tagged wild type (WT) or inactive mutant H532A TiPARP (HA). Relative luciferase activities were normalized by co-transfected Renilla-luciferase. (B) HIF-1α reporter activity measured in hypoxic HEK 293T cells transfected with vector, Flag-WT TiPARP or Flag-HA TiPARP. (C) qRT-PCR analysis of HIF target gene induction in response to hypoxia in HCT116 cells stably expressing control shRNA (Control) or shTiPARP (KD). The ratio of hypoxic to normoxic gene expression is shown. (D) Hypoxic induction of HIF target genes in Tet-on-inducible HCT116 cells measured by qRT-PCR. Expression of Flag-TiPARP was induced by treatment of 10 μM doxycycline for 24 hrs. In control group, cells were treated with DMSO. Error bars in A, B represent s.d. of six (A) or three (B) technical replicates from a representative experiment out of three independent experiments with similar results. In C, D, mean and s.d. of three independent experiments are shown. *p<0.05; **p<0.001; *p<0.0001; N.S.=not significant.

FIGS. 3A-3D. TiPARP interacts with and ADP-ribosylated HIF-1α, (A) Immunoprecipitation with anti-flag resins (top) or anti-HA resin (bottom) of HEK 293T cell lysate co-transfected with Flag-TiPARP and HA-HIF1α. Expression of HA-HIF1α and Flag-TiPARP in the input are shown in the right panel. (B) Schematic representations of full length and truncated HA-tagged HIF1α constructs. Colored boxes represent the functional domains of HIF-1α. Numbers refer to the truncation positions. (C) Co-immunoprecipitation of Flag-TiPARP and truncated HA-HIF-1α in HEK 293T cells. Expression of HA-tagged HIF-1α truncations in whole cell lysates are shown in the right panel. (D) Western blot analysis of mono-ADP-ribosylation on GFP-HIF-1α purified from HEK 293T cells co-transfected with empty vector (EV), Flag-tagged wild type TiPARP (WT), or inactive H532A mutant (HA).

FIGS. 4A-4D. TiPARP targets HIF-1α to specific nuclear bodies (TiPARP nuclear bodies) and negatively regulates the protein level of HIF-1α. (A) Confocal imaging showing that TiPARP is localized to spherical nuclear bodies and recruit HIF-1α. (B) Western blot of HIF-1α in HCT116 treated with negative control siRNA (siCtrl) or TiPARP siRNA (siTiPARP) with two distinct sequences, (C) Western blot of HIF-1α in HAP-1 wild type (WT) or TiPARP knockout (KG) cells. (D) Western blot of HIF-1α in HCT116 cells stably overexpressing empty vector (EV), Flag tagged wild type TiPARP (WT), or Flag tagged inactive H532A mutant TiPARP (HA). Hypoxia: 1% O₂; DMOG: 1 mM dimethyloxalylglycine as hypoxia mimics.

FIGS. 5A-5H. TiPARP represses Warburg effect and tumorigenesis. (A) Left: growth curves of luciferase control knockdown (Ctrl) and TiPARP knockdown (KD) HCT116 cells. Right: growth curves of control (Ctrl) and doxycycline-induced TiPARP expressing (Dox) HCT116 cells. Relative cell numbers were normalized to that of day 1. Error bars represent s.d. of three independent experiments. (B) Anchorage-independent growth assay of control knockdown (Ctrl) and TiPARP knockdown (KD) HCT116 cells (left), and inducible HCT116 cells treated with DMSO (Ctrl) or 10 μM doxycycline to induce vector or TiPARP expression (right). Colony numbers in each well of a 6-well plate were counted and shown as mean±s.e.m (three independent experiments), (C) Lactate production and glucose consumption of control and TiPARP knockdown HCT116 cells cultured in hypoxia for 24 hr. Values were normalized to normoxic controls. Mean and s.e.m are from three independent experiments. (D) Xenograft tumor growth of HCT116 ceils with doxycycline-inducible TiPARP over-expression (n=18). (E) Xenograft tumor growth of control (Ctrl) and TiPARP knockdown (KD) HCT116 cells (n=8). (F) Xenograft tumor growth of control (Ctrl) and TiPARP knockdown (KD) MCF-7 xenografts (n=9). In d-f, error bars represent standard error of the mean (s.e.m.) (G) Immunohistochemical analysis of CD31 expression in HCT116 xenografts. Image of one representative pair was shown. Vascular distribution in tumors was quantified by counting CD31-positive microvessels per ×20 field (n=8) and shown as mean mean±s.e.m. Scale bar, 200 μm. (H) Kaplan-Meier survival curve of 3951. (left) and 157 (right) breast cancer patients enrolled in TCGA, GEO and EGA database, analyzed by miRpower and PROGgene. Patients were divided into two groups (top and bottom 50% TiPARP expression) based on TiPARP mRNA levels in their tumors. NS, not significant, *p<0.05; **p<0.01; ***p<0.001.

FIG. 6. Proposed model depicting the negative feedback loop regulation of HIF-1α via TiPARP nuclear bodies.

FIGS. 7A-7C. TiPARP is a target gene and negative regulator of HIF-2α, (A) Top: RT-PCR analysis of TiPARP mRNA. Bottom: western blot of FTA-HIF-2α in HEK 293T cell lysate with HA antibody. (B) Luciferase reporter assay for HIF-2α transactivation in HEK 293T cells co-transfected with HA-HIF-2α and wild type TiPARP (WT)/catalytic inactive H532A mutant (HA). Results are presented as mean±s.d. (three independent experiments). (C) Immunoprecipitation of HEK 293T cell lysate with anti-flag resins. Cells were co-transfected with Flag-TiPARP and HA-HIF-1α/HIF-2α. Expression levels of HA-HIF-1α/HIF-2α in the whole cell lysates are shown at the bottom.

FIGS. 8A-8G. TiPARP is a tumor suppressor. (A) Expression of HIF-1α target genes in RCC4 cells measured by qRT-PCR. (B) Growth curve of control luciferase knockdown (Ctrl) and TiPAPR knockdown (KD) MCF-7 cells. (C) Representative images of transwell migration assays in different cancer cell lines stably expressing luciferase shRNA (Ctrl) or TiPAPR shRNA (KD). (D) Lactate secretion and glucose uptake of control and TiPARP knockdown RCC4 cells. (E) mRNA level of HIF-1α target genes in MCF-7 xenografts. (F) Representative images of CD31 immunohistochemical analysis in MCF-7 xenografts. Scale bar, 300 μm. (G) Immunohistochemistry staining of HIF-1α and TiPARP in HCT116 xenografts. Arrows point to representative sites of HIF-1α and TiPARP nuclear staining. Scale bar, 200 μm. Error bars represent s.d. (three independent experiments) except in d, which indicate s.e.m.

FIGS. 9A-9G. (A) Left panel: representative confocal images of GFP-tagged mono-ADP-ribosylation detection (MAD) probe in HeLa cells. Scale bar, top: 5 μm; bottom: 10 μm. Right panel: confocal imaging of GFP-MAD co-transfected with Flag-TiPARP H532A (inactive mutant) in HeLa cells. Scale bar, 5 μm. (B) Co-localization of GFP-MAD with wild type Flag-TiPARP in HeLa cells. Scale bar, top: 5 μm, bottom: 10 μm. (C) Representative confocal image of flag-cMyc co-transfected with vector (first row) or GFP-TiPARP (bottom two rows). Scale bar, 5 μm. (D) Transcription activity of c-Myc was measured by luciferase reporter with c-Myc binding sites. c-Myc was co-transfected with wild-type (WT) TiPARP or catalytically inactive H532A mutant (HA) in HeLa cells. Mean and s.d. from two independent experiments are shown, (e) qRT-PCR analysis of TiPARP mRNA expression in MCF-7 cells treated with DMSO or 10 nM β-estradiol for 24 hr. (F) Transactivation of estrogen receptor α (ERα) and/or estrogen receptor β (ERβ) measured by luciferase reporter containing estrogen response elements (EREs). HEK 293T cells were transfected with Flag-TiPARP, together with HA-ERα and/or Flag-ERβ. Mean and s.d correspond to three or four technical replicates from a representative experiments. (G) Immunofluorescence staining of HA-ERα and Flag-TiPARP with HA and Flag antibodies in HeLa cells. Scale bar, 5 μm.

FIGS. 10A-10E. (A) Overexpression of active TiPARP decreases cMyc protein levels. Wild type (active) or inactive HA mutant of TiPARP was expressed in HCT 116C cells. WT overexpression significantly decreased cMyc levels. EV: empty vector, WT: wild type (active) TiPARP over-expression, HA: inactive mutant TiPARP over-expression. (B) TiPARP regulates cMyc protein levels by regulating the protein degradation pathway. 20 μM MG132 (protease inhibitor) treatment over 2 hours effectively blocked the effect of TiPARP overexpression in cells. EV: empty vector, WT: wild type (active) TiPARP over-expression, HA: inactive mutant TiPARP over-expression. (C) Overexpression of active TiPARP (WT) caused a decrease in ERα, protein levels, whereas overexpression of the inactive mutant (HA) had no effect in HCT116 cells. EV: empty vector, WT: wild type (active) TiPARP over-expression, HA: inactive mutant TiPARP over-expression. (D) Knockdown of TiPARP in HCT116 cells caused ERα protein levels to increase. (E) Knockdown of TiPARP in MCF-7 cells caused ERα protein levels to increase.

FIG. 11. A compounds's ability to inhibit cancer cell viability correlates with the compound's ability to induce TiPARP mRNA. The experiments were carried out in three different cell lines (SKBR3, MCF7, and HCT116). Each dot in the figure represents a compound, including Biochanin A, diindolylmethane, 4-hydroxytamoxifen and tamoxifen. This correlation shows the ability to induce TiPARP is important for a given compounds' anti cancer activity.

### DETAILED DESCRIPTION

### Definitions

As used herein, the term “about” refers to an approximately ±10% variation from a given value.

The terms “anticancer” or “anti-tumor” refer to a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size and mass of a tumor during therapy.

The term “expression” refers to the process of converting genetic information of a polynucleotide into RNA through transcription, which is catalyzed by an enzyme, RNA polymerase and into protein, through translation of mRNA on ribosomes. Expression can be, for example, constitutive or regulated, such as, by an inducible promoter (e.g., lac operon, which can be triggered by Isopropyl β-D-1-thiogalactopyranoside (IPTG)). Up-regulation or overexpression refers to regulation that increases the production of expression products (mRNA, polypeptide or both) relative to basal or native states, while inhibition or down-regulation refers to regulation that decreases production of expression products (mRNA, polypeptide or both) relative to basal or native states.

The term “gene,” as used herein, refers to a segment of nuclei c acid that encodes an individual protein or RNA and can include both exons and introns together with associated regulatory regions such as promoters, operators, terminators, 5′ untranslated regions, 3′ untranslated regions, and the like.

The term “prevention” used herein means delay or eliminate the onset of cancer, or reduce the occurrences of cancer among a population of patients.

A “therapeutically effective dose” or “therapeutic dose” is an amount sufficient to effect desired clinical results (i.e., achieve therapeutic efficacy). For example, a therapeutically effective dose of an agent that activates TIPARP activity refers to an amount that, when administered as described herein, brings about a positive therapeutic response, such as an amount having anti-tumor activity. A positive therapeutic response may include preventing or delaying progression of a tumor. A therapeutically effective dose can be administered in one or more administrations. For purposes of this disclosure, a therapeutically effective dose of an agonist of TiPARP and/or compositions (e.g., compositions that include an agonist of TiPARP) is an amount that is sufficient, when administered to the individual, to palliate, ameliorate, stabilize, reverse, prevent, slow or delay the progression of the disease state (e.g., cancer, tumor, etc.) by, for example, inducing TiPARP activity or protein levels.

### General Description

An aspect of the present disclosure is predicated at least in part on upregulating (e.g., activating) tetrachlorodibenzo-p-dioxin (TCDD) inducible PARP (TiPARP or ARTD14) for therapeutic purposes in the prevention, amelioration and/or attenuation of cancer. The present disclosure is based on the finding that upregulating (e.g., activating) TiPARP is an effective anticancer method. The inventors have found that upregulating TiPARP activity impedes cell growth and metabolic reprogramming cancer cells through the inhibition of HIF-1 signaling.

### TiPARP Agonists

In some embodiments, upregulating TiPARP is achieved by a TiPARP agonist. A TiPARP agonist activates TIPARP activity directly, or leads to an elevation of TiPARP protein levels.

In some embodiments, the TiPARP agonist is an agent that activates TiPARP ADP-ribosylation activity. An agent that activates TiPARP ADP-ribosylation activity is any agent that increases or otherwise modulates the activity of a TiPARP ADP-ribosylate enzyme. In some embodiments, the TiPARP agonist interacts with TiPARP directly to activate TiPARP enzymatic activity.

In some embodiments, the TiPARP agonist leads to an elevated expression of the TiPARP gene. In some embodiments, an elevated expression refers to at least 150% of protein activity or amount as compared with an appropriate endogenous control. Elevated expression can be achieved by mutating the protein to produce a more active form or a form that is resistant to inhibition, by removing inhibitors, or adding agonists, and the like. Elevated expression can also be achieved by removing repressors, adding multiple copies of the gene to the cell, or upregulating the endogenous gene, and the like. In a specific embodiment, in order to achieve elevated expression in amount of a protein, one or more expression vectors encoding the protein is added to the cell. In some embodiments, elevated expression of TiPARP is constitutive. In some embodiments, elevated expression of TiPARP is transient or inducible.

In some embodiments, the TiPARP agonist is a small molecule compound. The term “small molecule compound” herein refers to small organic chemical compound, generally having a molecular weight of up to or less than 5000 daltons, 2000 daltons, 1500 daltons, 1000 daltons, 800 daltons, or 600 daltons, or a molecular weight within a range bounded by any two of the foregoing values.

In a first set of embodiments, the TiPARP agonist is tamoxifen or a tamoxifen derivative (i.e., selected from “tamoxifen compounds”). The tamoxifen compounds can be conveniently described by the following generic formula:

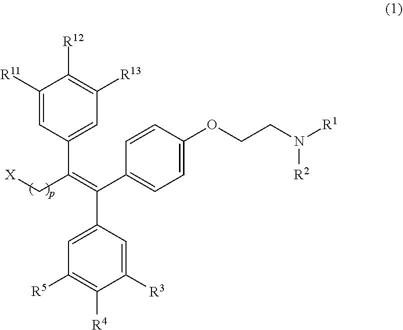

In the above Formula (1), R¹ and R² are independently selected from alkyl groups containing one to three carbon atoms. The alkyl groups may be straight (linear), branched, or cyclic. Examples of alkyl groups for R¹ and R² include methyl, ethyl, n-propyl and isopropyl. R¹ and R² may alternatively interconnect to form a five-membered or six-membered heterocycloalkyl ring, such as a pyrrolidinyl, piperidinyl, 4-methylpiperidinyl, piperazinyl, or morpholinyl ring. In Formula (1), R³, R⁴, and R⁵ are independently selected from hydrogen atom, halogen atom (e.g., F, Cl, Br, and/or I), methyl, ethyl, hydroxy (OH), methoxy (—OCH₃), and ethoxy (—OCH₂CH₃). In some embodiments, R³ and R⁵ are hydrogen atoms, and R⁴ is selected from halogen atom, methyl, ethyl, hydroxy, methoxy, and ethoxy, or R⁴ is selected from hydroxy, methoxy, and ethoxy. In Formula (1), R¹¹, R¹², and R¹³ are independently selected from hydrogen atom, hydroxy, and methoxy groups. In some embodiments, at least one of R¹¹, R¹², and R¹³ is a hydroxy or methoxy group. In other embodiments, R¹¹ and R¹³ are hydrogen atoms and R¹² is a hydroxy or methoxy group. In Formula (1), X is a hydrogen atom or halogen atom, and p is 2 or 3. A variety of tamoxifen derivatives, within the scope of Formula (3), are described in detail in, for example, U.S. Pat. No. 4,839,155, EP 0260066, and WO 1996040616, the contents of which are herein incorporated by reference in their entirety. Formula (1) and sub-formulas thereof are also intended to include all pharmaceutically acceptable salt forms.

In specific embodiments of Formula (1), X is hydrogen and p is 1, which results in the tamoxifen compound having the following structure:

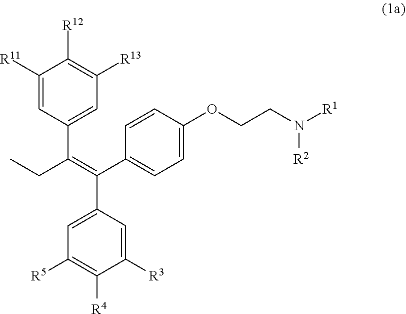

In more specific embodiments of Formula (1a), R¹ and R² are independently selected from alkyl groups containing one to three carbon atoms. In further specific embodiments of Formula (1a), R¹ and R² are methyl groups, which corresponds to the following structure:

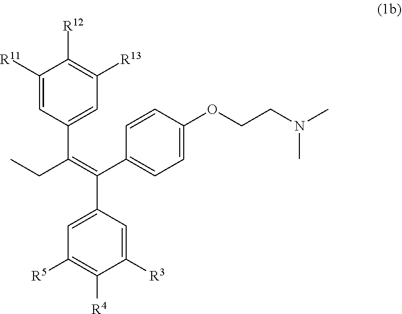

In other embodiments, in any of Formulas (1), (1a), or (1b), R³ and R⁵ are hydrogen atoms and R⁴ is selected from the group consisting of halogen atom, methyl, ethyl, hydroxy (OH), methoxy (—OCH₃), and ethoxy (—OCH₂CH₃), or R⁴ is selected from the group consisting of hydroxy (OH), methoxy (—OCH₃), and ethoxy (—OCH₂CH₃), or R⁴ is hydroxy.

In more specific embodiments of Formula (1b), R³, R⁴, and R⁵ are hydrogen atoms, which corresponds to the following structure (tamoxifen itself):

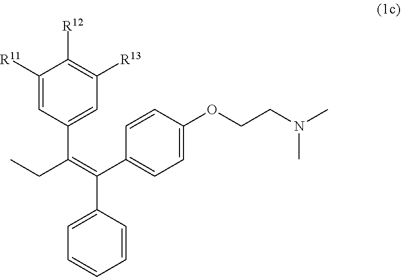

In some embodiments of Formula (1c), at least one of R¹¹, R¹², and R¹³ is a hydroxy or methoxy group. In more specific embodiments, R¹¹ and R¹³ are hydrogen atoms and R¹² is a hydroxy or methoxy group. In a further particular embodiment, R¹² is hydroxy, in which case the tamoxifen compound is 4-hydroxytamoxifen, which corresponds to the following structure:

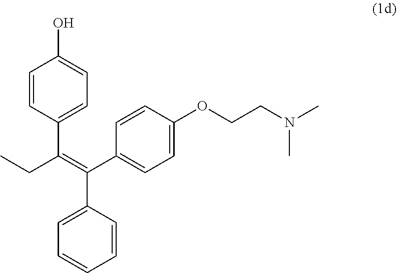 [SMILES: CC/C(=C(\c1ccccc1)c1ccc(OCCN(C)C)cc1)c1ccc(O)cc1]

In other embodiments, the tamoxifen compound can be within any of the following generic structures, wherein R¹, R², R³, R⁴, and R⁵ are as defined above, including all selections, sub-selections, and specific examples earlier provided

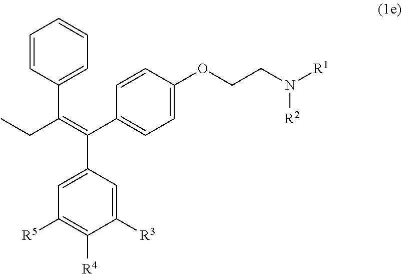

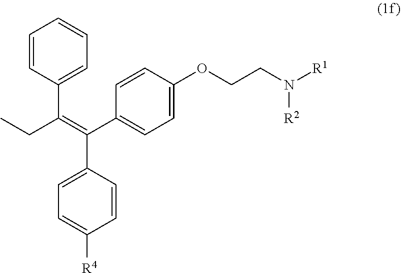

In a second set of embodiments, the TiPARP agonist is a flavone or isoflavone compound within the following generic formula:

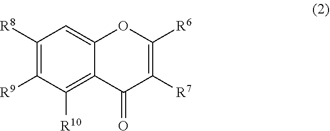

In Formula (2) above, R⁶ and R⁷ are independently selected from (i) hydrogen atom and (ii) phenyl ring optionally substituted with one or two OH and/or methoxy (OCH₃) groups, provided that one of R⁶ and R⁷ is (ii). In Formula (2), R⁸, R⁹, and R¹⁰ are independently selected from hydrogen atom, methyl, phenyl, hydroxy, and methoxy groups, wherein the phenyl is optionally substituted with a hydroxy or methoxy group; wherein R⁸ and R⁹ may optionally interconnect as a benzene ring, or R⁹ and R¹⁰ may optionally interconnect as a benzene ring.

In some embodiments of Formula (2), precisely or at least one of R⁸, R⁹, and R¹⁰ is a hydroxy group or methoxy group and none of R⁸, R⁹, and R¹⁰ interconnect. In more specific embodiments, precisely or at least two of R⁸, R⁹, and R¹⁰ are hydroxy groups or methoxy groups. In further embodiments, one of R⁶ and R⁷ is a phenyl ring substituted with a hydroxy or methoxy group, wherein the hydroxy or methoxy group is typically-located on the meta or para position of the phenyl ring.

Some examples of flavone or isoflavone TiPARP agonist compounds include the following:

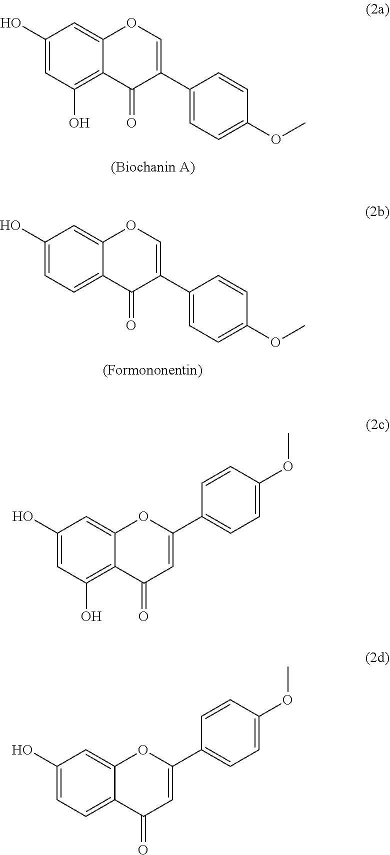 [SMILES: COc1ccc(-c2coc3cc(O)cc(O)c3c2=O)cc1 ; COc1ccc(-c2coc3cc(O)ccc3c2=O)cc1 ; COc1ccc(-c2cc(=O)c3c(O)cc(O)cc3o2)cc1 ; COc1ccc(-c2cc(=O)c3ccc(O)cc3o2)cc1]

In other embodiments of Formula (2), R⁹ and R¹⁰ interconnect as a benzene ring and R⁸ is a hydrogen atom, which corresponds to the following structure:

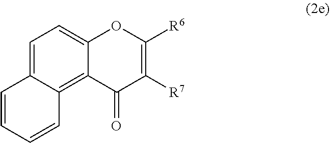

In specific embodiments of Formula (2c), one of R⁶ and R⁷ is an unsubstituted phenyl ring. In particular embodiments of Formula (2c), the TiPARP agonist has the following structure, which corresponds to beta-naphthoflavone (BNF, DB06732, also known as 5,6-benzoflavone):

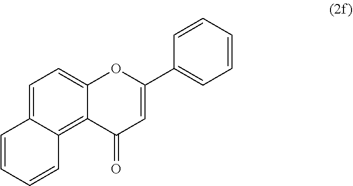 [SMILES: O=c1cc(-c2ccccc2)oc2ccc3ccccc3c12]

Notably, the above flavone and isoflavone types of TiPARP agonists may more specifically function as aryl hydrocarbon receptor (AHR) agonists or an estrogen receptor (ER) agonist.

In a third set of embodiments, the TiPARP agonist is an indolyl-containing compound, which contains one, two, or more indole rings. Generally, the indolyl nitrogen is not substituted in such compounds (i.e., the indolyl nitrogen is attached to a hydrogen atom). The one or more indolyl rings are also generally substituted at least in the 3-position, and may or may not be substituted on the 4-, 5-, 6-, or 7-positions. The substituent is generally either a connecting moiety between indolyl rings (typically containing one, two, three, or four linking atoms, e.g., methylene, dimethylene, —O—, —NH—, —CH₂—O—CH₂—, or —CH₂—NH—CH₂—) or a non-linking alkyl group, such as methyl, ethyl, n-propyl, or n-butyl that may or may not be substituted by a heteroatom-containing group, such as —OH, —OCH₃, —NH₂, —NHCH₃ or —N(CH₃)₂. In some embodiments, the heteroatom-containing group functions as an endcapping group on the alkyl group (e.g., —CH₂OH or —CH₂CH₂OH). The indolyl-containing compound may also be a pharmaceutically acceptable salt form thereof.

Some examples of indolyl-containing TiPARP agonist compounds include the following:

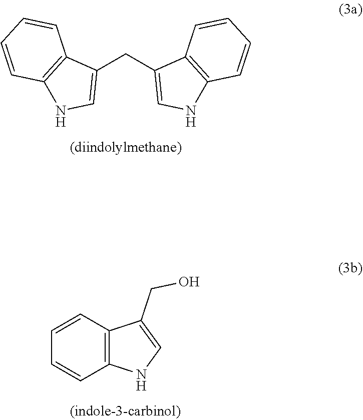 [SMILES: c1ccc2c(Cc3c[nH]c4ccccc34)c[nH]c2c1 ; OCc1c[nH]c2ccccc12]

In a fourth set of embodiments, the TiPARP agonist is a chlorinated dibenzo-p-dioxin (CDBD) derivative with the following chemical formula:

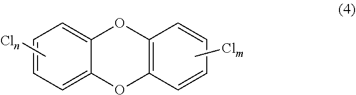

In the above Formula (4), n represents a number between 0 and 4, and m independently represents a number between 0 and 4, provided that the sum of n and m (i.e., n+m) is at least 1, 2, 3, or 4.

In a specific embodiment, the CDBD derivative is tetrachlorodibenzo-p-dioxin (TCDD) having the following chemical formula:

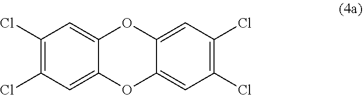 [SMILES: Clc1cc2c(cc1Cl)Oc1cc(Cl)c(Cl)cc1O2]

In a fifth set. of embodiments, the TiPARP agonist is a hydroxylated or methoxylated stilbene compound. The stilbene compound may contain precisely or at least one, two, three, four, five, or six hydroxy groups, or precisely or at least one, two, three, four, five, or six methoxy groups, or a combination of hydroxy and methoxy groups. The stilbene derivative may or may not also include one, two, or more halogen atoms (e.g., F, Cl, or Br). Some examples of stilbene derivatives include:

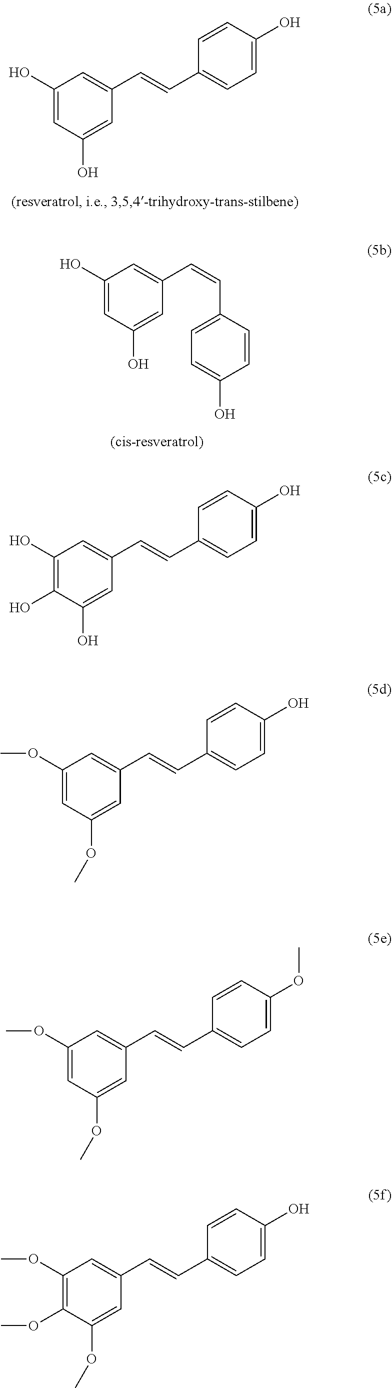

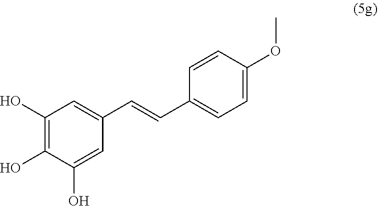 [SMILES: COc1ccc(/C=C/c2cc(O)c(O)c(O)c2)cc1]

A large number of derivatives of stilbenes, including derivatives of cis- and trans-resveratrol, in particular, are known, including methoxylated and/or halogenated versions thereof such as described in W. Nawaz et al., Nutrients, 9(11), 1188, November 2017, the contents of which are herein incorporated by reference.

In a sixth set of embodiments, the TiPARP agonist is a benzimidazole derivative, which may be a proton pump inhibitor, such as omeprazole or derivative thereof. A derivative of omeprazole may contain, for example, a substituted (e.g., methylated or ethylated) nitrogen on the benzimidazole ring system. The benzimidazole derivative may also be a pharmaceutically acceptable salt form.

Omeprazole has the following chemical formula:

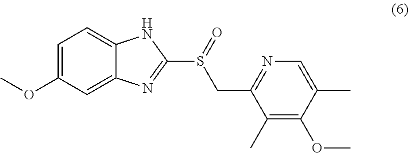

### Administration

In some embodiments, a TiPARP agonist is administered to the subject continuously. In one embodiment, a TiPARP agonist is not administered to the subject continuously; rather it is administered intermittently. In a specific embodiment, intermittent TiPARP agonist administration is performed once every other day, every three days, every four days, every five days, or once a week. In another specific embodiment, intermittent TiPARP agonist administration is performed once every hour, every two hours, every three hours, every six hours, every ten hours, or every twelve hours.

In some embodiments, a therapeutically effective amount of a TiPARP agonist is about 0.2 mg/kg to 100 mg/kg. In other embodiments, the effective amount of a TiPARP agonist is about 0.2 mg/kg, 0.5 mg/kg, 1 mg/kg, 8 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 150 mg/kg, 175 mg/kg or 200 mg/kg of TiPARP agonist.

Typically, the TiPARP agonist is administered as a solution or suspension of the TiPARP agonist in a pharmaceutically acceptable carrier. For purposes of this disclosure, the term “pharmaceutically acceptable carrier” refers to any of the accepted pharmaceutical carriers known in the art. The carrier may be, for example, a phosphate buffered saline solution or those suitable for use in tablets, granules, capsules, and the like. Typically, solid carriers contain excipients, such as starch, milk, sugar, certain types of clay, gelatin, stearic acid or salts thereof, magnesium or calcium stearate, talc, vegetable fats or oils, gum, glycols or other known excipients. Such carriers may also include flavor and color additives or other ingredients. Compositions comprising such carriers are formulated by well-known conventional methods. The TiPARP agonist can be admixed with a pharmaceutically acceptable carrier to make a pharmaceutical preparation in any conventional form including, inter alia, a solid form, such as tablets, capsules (e.g. hard or soft gelatin capsules), pills, cachets, powders, granules, and the like; a liquid form such as solutions, suspensions, or in micronized powders, sprays, aerosols and the like.

The TiPARP agonist may be administered by any suitable mode, such as by injection. In different embodiments, the TiPARP agonist may be administered by different routes of administration such as oral, oronasal, or parenteral route. “Oral” or “peroral” administration refers to the introduction of a substance into a subject's body through or by way of the mouth and involves swallowing or transport through the oral mucosa (e.g., sublingual or buccal absorption) or both. “Oronasal” administration refers to the introduction of a substance into a subject's body through or by way of the nose and the mouth, as would occur, for example, by placing one or more droplets in the nose. Oronasal administration involves transport processes associated with oral and intranasal administration. “Parenteral administration” refers to the introduction of a substance into a subject's body through or by way of a route that does not include the digestive tract. Parenteral administration includes subcutaneous administration, intramuscular administration, transcutaneous administration, intradermal administration, intraperitoneal administration, intraocular administration, and intravenous administration.

### Cancer Types

In some embodiments, the disclosure is directed to treating cancers that show increased HIF-1 signaling. In some embodiments, the increased HIF-1 signaling in cancer cells is associated with elevated expression of HIF-1α expression as compared to normal (non-tumor) cells of the same type.

In specific embodiments, the cancer selected from the group consisting of breast cancer, colon cancer, lung cancer, skin cancer, brain cancer, blood cancer, cervical cancer, liver cancer, prostate carcinoma, pancreas carcinoma, gastric carcinoma, ovarian carcinoma, renal cell carcinoma, mesothelioma, and melanoma. In some embodiments, the cancer being treated is not breast cancer, particularly when the TiPARP agonist is selected from any of Formulas (1)-(1d). For example, the cancer being treated may be colon or lung cancer when using a TiPARP agonist selected from any of Formulas (1)-(1f).

### Expression Vectors

In yet another aspect, this disclosure provides an expression vector comprising a nucleotide sequence encoding an exogenous TiPARP gene, operably linked to a regulatory region that is functional in a cell. The term “exogenous,” as used herein, refers to a substance or molecule originating or produced outside of an organism. The term “exogenous gene” or “exogenous nucleic acid molecule,” as used herein, refers to a nucleic acid that codes for the expression of an RNA and/or protein that has been introduced (“transformed”) into a cell or a progenitor of the cell. An exogenous gene may be from a different species (and so a “heterologous” gene) or from the same species (and so a “homologous” gene), relative to the cell being transformed. A transformed cell may be referred to as a recombinant or genetically modified ceil. An “endogenous” nucleic acid molecule, gene, or protein can represent the organism's own gene or protein as it is naturally produced by the organism.

In some embodiments, the regulatory region comprises an inducible promoter or a tissue-specific promoter. In a specific embodiment, the inducible promoter is a tet-on promoter.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1: Materials and Methods

### Cell Culture, Hypoxic Incubation and Transfection

MCF-7, RCC4 and HEK293T cells were obtained from the American Type Culture Collection (Manassas. Va.), and were cultured in Dulbecco's modified Eagle's medium (Gibco, 11965-092) supplemented with 10% heat-inactivated fetal bovine serum (FBS, Gibco, 26140079). HCT116 cells were cultured in McCoy's 5A medium (Ser. No. 16/600,082) with 10% FBS. Cells were grown in a humidified atmosphere at 37° C. at gas tensions of 20% O₂/5% CO₂ for normoxic incubation and 1% O₂/5% CO₂ for hypoxic incubation. HIF-1α in cell lysates was detected by western blot with HIF-1α antibody (BD Biosciences).

For transient overexpression of proteins of interest, cells were transfected with expression vectors using FuGene 6 (Promega, E2691) according to the manufacturer's protocol. To stably knock down TiPARP, lentivirus was produced by transfecting HEK 293T cells with pCMV-ΔR8.2 (packaging vector), pM2D.G (envelope vector) and shRNAs (Sigma). Virus-containing medium was harvested 24 hr after transfection. Target ceils were seeded in a 6-well plate 20 hr prior to transduction (3×10⁵ cells per well). At the confluence of 50%, target cells were transduced with virus particles in medium supplemented with 5 μg/ml polybrene (Sigma), and incubated for 6 hr. Remove supernatant and add fresh medium to the cells. 72 hr after infection, 2 μg/mL puromycin was added to the cell culture media for 10 days to select stably transduced target cells.

### Plasmids

Human TIPARP cDNAs was amplified by PCR using pCMV6-XL4-hTiPARP as templates (Origene, Rockville, Md., USA. Cat: RC230398). Following amplification, the CDS of TIPARP was cloned into EcoRI and SalI sites in pAcGFP-C1 vector to generate GFP-TiPARP expression vector (forward primer: 5′-AGTCAGGAATTCTATGGAAATGGAAACCACCGA ACCTGAGCCAGA-3′ (SEQ ID NO: 2); reverse primer: 5′-AGTCAGGGATCCCTACTTATCGTCGTCATCCTTGTAATCCAGGATATCATTTGC-3′) (SEQ ED NO: 3). Expression vectors for TiPARP H532A catalytic mutant was generated by QuikChange site-directed mutagenesis (forward primer: 5′-AAATGAGAGACATTTATTTGCTGGAACATCCCAGGAT GTGGT-3′ (SEQ ID NO: 4); reverse primer: 5′-AAATAAATGTCTCTCATTTATTATCCTGTCACGGCCAAACATTTTCC-3′) (SEQ ID NO: 5). Luciferase reporter construct with TiPARP promoter was obtained by PCR amplification of a 1.2 kb proximal promoter fragment from human cDNA library and inserting this fragment into the Xho I and Hind III restriction sites in the pGL4.14 vector (Promega) (forward primer: 5′-AGTCAGCTCGAGAGATCTTGTCTCAA TAAGATTTTAAATGTAAAGATTTTCA-3′ (SEQ ID NO: 6); reverse primer: 5′-AGTCAGAAGCTTGTGCGGTGGACTTATGCTCCC-3′ (SEQ ID NO: 7)). Mutant promoter-luciferase construct was obtained by QuikChange site-directed mutagenesis on core HRE sequence (forward primer: 5′-TCCTTCCTCACAGCCTTGTGTAGACGCGGACCC-3′ (SEQ ID NO: 8); reverse primer: 5′-GGCTGTGAGGAAGGAAGGCGCGTGCCGCGTGGG-3′ (SEQ ID NO: 9)). pcDNA-HA-HIF-1α plasmid was a gift from Dr. M. Celeste Simon. Truncations of N-terminal HA tagged HIF-1α was obtained by introducing stop codons at truncated sites via mutagenesis (region 1-70 forward primer: 5′-TATTTGCGTGTGAGGTAACTTCTGGATGC-3′ (SEQ ID NO: 10); reverse primer: 5′-AAGTTACCTCACACGCAAATAGCTGATGGTAAG-3′ (SEQ ID NO: 11). Region 1-158 forward primer: 5′-GCCTTGTGAAAAAGGGTTAAGAACAAA ACACAC-3′ (SEQ ID NO: 12); reverse primer: 5′-TTCTTAACCCTTTTTCACAAGGCCATT TCTGTGT-3′ (SEQ ID NO: 13). Region 1-298 forward primer: 5′-ATGATATGTTTACTAAAGGATAAGTCACCACAGG-3′ (SEQ ID NO: 14); reverse primer: 5′-GACTTATCCTTTAGTA AACATATCATGATGAGTTTT-3′ (SEQ ID NO: 15). Region 1-575 forward primer: 5′-GACTTCCAGTTACGTTAATTCGATCAGTTGTCA-3′ (SEQ ID NO: 16); reverse primer: 5′-GAATTAACGTAACTGGAAGTCATCATCCATTGG-3′ (SEQ ID NO: 17). Region 1-603 forward primer: 5′-GTTACAGTATTCCAGTAGACTCAAATACAAGAACC-3′ (SEQ ID NO: 18); reverse primer: 5′-AGTCTACTGGAATACTGTAACTGTGCTTTGAG-3′ (SEQ ID NO: 19). Region 1-785 forward primer: 5′-TAGACTGCTGGGGCAATAAATGGATGAAAG-3′ (SEQ ID NO: 20); reverse primer: 5′-CATTTATTGCCCCAGCAGTCTACATGCTAAAT-3′ (SEQ ID NO: 21)). Human HIF-1α cDNA ORF clone with C-GFPSpark® tag was purchased from Sino Biological (Cat #HG11977-ACG). HRE-luciferase construct was obtained from Navdeep Chandel (#26731); pUltra-dox (#58749) and pUltra-puro-RTTA3(#58750) constructs were obtained from Yildirim Dogan and Kitai Kim; HA-HIF2α-pcDNA3(#18950) was obtained from William Kaelin; pBV-Luc wt MBS1-4 (#16564) was obtained from Bert Vogel stein; pcDNA-HA-ERapha WT (#49498) was obtained from Sarat Chandarlapaty; pcDNA-Flag-ERbeta (#35562) was obtained from Harish Srinivas; 3× ERE-TATA-luc (luciferase reporter containing three copies of estrogen response elements) (#11354) construct was obtained from Donald McDonnell via Addgene. Human c-Myc expression construct with N-terminal Flag-tag was obtained by PCR amplification from cDNA library and subcloning into pCMV-tag-4a vector through BamHI and XhoI sites. GFP tagged macrodomains (mono-ADPribosyiation detection probe, GFP-MAD) was performed as described previously (Lanczky, A. et ah, Breast Cancer Res Treat 160. 439-446, (2016)) with slight modifications. Macrodomains 1-3 of human PARP14 was amplified from cDNA library and subcloned into pAcGFP-C1 vector via XhoI and BamHI sites. Human cMyc-cDNA with N-terminal Flag-tag was obtained by PCR amplification of Flag-c-Myc and subcloning via BamHI and XhoI sites into pCMV-tag-4a vector. shRNA plasmids targeting human TiPARP were purchased from Sigma. Sequences for human TIPARP shRNAs are 5′-CCGGAGGTCTTTGAGGCCAATATTACTCGAGTAATATTGGCCTCAAAGACCTTT TTTG-3′ (SEQ ID NO: 22) (sh1) and 5′-CCGGGAAGGCAAGCTACTCTCATAACTCGAGTTATGAGAGTAGCTTGCCTTCT TTTTG-3′ (SEQ ID NO: 23) (sh2).

Quantitative Real Time PCR (qRT-PCR)

Total RNA. was isolated using RNeasy Mini kit (Qiagen) according to the manufacturer's instructions. 0.2 μg sample of total RNA was employed for cDNA synthesis using the Superscript III First-Strand Synthesis kit (Invitrogen) following the manufacturer's instructions. For quantitative real-time PCR analysis, iTaq Universal SYBR Green Supermix (Bio-Rad) was used following the manufacturer's instructions. The reaction was performed with QuantStudio™ 7 Flex Real-Time PCR System (APPLIED BIOSYSTEMS™). In each run, melt curve analysis was performed to ensure the amplification of a single product. The relative expression of each gene, normalized to actin, was calculated using the 2^{-ΔΔCt} method. The following primers were used for qRT-PCR: VEGF: 5′-CTACCTCCACCATGCCAAGT-3′ (SEQ ID NO: 24) and 5′-GCAGTAGCTGCGCTGATAGA-3′ (SEQ ID NO: 25); GLUT1: 5′-CGGGCCAAGAGTGTG CTAAA-3′ (SEQ ID NO: 26) and 5′˜TGACGATACCGGAGCCAATG-3′ (SEQ ID NO: 27); PDK1: 5′-ACCAGGACAGCCAATACAAG-3′ (SEQ ID NO: 28) and 5′-CCTCGGTCACTCATCTTCAC-3′ (SEQ ID NO: 29); WSBJ: 5′-CGTACTATAGGTGAACTTTTAGCTCCT-3′ (SEQ ID NO: 30) and 5′-CCAAAGGAAAACTGCTTTACTGG-3′ (SEQ ID NO: 31); LDHA: 5′-CTCCTGTGCAAAATGCCAAC-3′ (SEQ ID NO: 32) and 5′-CCTAGAGCTCACTAGTCA CAG-3′ (SEQ ID NO: 33); CXCR4: 5′-TGGGTGGTTGTGTTCCAGTTT-3′ (SEQ ID NO: 34) and 5′-ATGCAATAGCAGGACAGGATGA-3′ (SEQ ID NO: 35); TIPARP: 5′-GGCAGATTTGAATGCCATGA-3′ (SEQ ID NO: 36) and 5′-TGGACAGCCTCCGTAGTTGGT-3′ (SEQ ID NO: 37), ACTIN: 5′-GATCATTGCTCCTCCTGAGC-3′ (SEQ ID NO: 38) and 5′-ACTCCTGCTTGCTGATCCAC-3′ (SEQ ID NO: 39).

### Luciferase Reporter Assays

HEK293T cells were seeded in 24-well plate and transfected using Fugene 6 (Promega) with 1 μg of the following plasmids as described in the text: pRL Renilla luciferase control reporter vector (Rluc), pGL4-HREx3, pcDNA-HA-HIF1α, pCMV6-flag-TIPARP wild type and pCMV6-flag-TIPARP H532A. 24 hr post transfection, cells were lysed and luciferase activity was determined using the Dual-Luciferase Reporter Assay system (Promega). In hypoxic groups, cells were transfected with Rluc, pGL4-HREx3 and wild type or mutant TiPARP. 12 hours post transfection, cells were switched to hypoxia culture condition (1% O₂) for 12 hours, followed by lysis and luciferase activity measurement.

### Co-Immunoprecipitation

To examine the interaction between FLAG-tagged TiPARP and HA-tagged HIF1α, HEK293T cells transfected with FLAG-TiPARP with empty vector or HA-HIF1α and cultured overnight. Cells were then collected and lysed in 1% NP-40 lysis buffer (150 mM NaCl, 25 mM Tris, 1% NP40, 10% glycerol, with protease inhibitor cocktail freshly supplemented). For each sample, 1 mg of whole cell lysate (quantified with Bradford regent) was incubated with 10 μL of anti-FLAG M2 affinity gel for 3 hr at 4° C. under constant mixing. The resulting affinity gel was washed three times with 1 mL IP washing buffer (50 mM NaCl, 25 mM Tris, 0.1% NP40) and heated in protein loading buffer at 95° C. for 10 min. Western blot was performed to detect the interaction of indicated proteins. Flag and HA tagged proteins were detected with HRP conjugated anti-flag antibody and anti-HA antibody respectively (Santa Cruz). Endogenous HIF-1β was detected with HIF-1β/ARNT antibody (Cell Signaling #5537).

### Immunofluorescence

Cells were seeded in 35 mm glass bottom dishes (MatTek) and transfected with FLAG-TiPARP, and GFP-HIF1α in HEK293T cells overnight. Cells were then rinsed with PBS and fixed with 4% paraformaldehyde in PBS for 15 min at room temperature. Fixed cells were washed twice with PBS, permeabilized with 0.1% saponin in PBS and blocked with 5% BSA for 30 min at room temperature. Cells were then incubated with indicated anti-flag antibody overnight at 4° C. in dark at 1:1000 dilution (in PBS with 0.1% saponin, 5% BSA). Cells were washed with PBS with 0.1% saponin for three times and incubated with Alexa Fluor 488 conjugated goat anti-mouse IgG secondary antibody or Cy3-conjugated goat anti-rabbit IgG secondary antibody at 1:1000 dilution (in PBS with 0.1% saponin) in the dark for 1 hr at room temperature. Samples were washed with PBS three times and mounted with Fluoromount-G (SouthernBiotech, 0100-01). Samples were imaged with Zeiss LSM880 inverted confocal microscopy. Images were processed with ZEN and Fiji softwares.

### Detection of ADP-Ribosylation

To detect mono-ADP-ribosylation on HIF1α, HEK293T cells were transfected with pcDNA-HA-HIF1α with pCMV6-FLAG-TIPARP WT/H532A or empty pCMV vector and cultured overnight. After harvesting cells, each group of cells (from one plate of 10 cm dish) was lysed with 100 μl of 4% SDS lysis buffer with nuclease and centrifuged at 17,000 rpm for 10 minutes to obtain clear lysate. Whole cell lysates were diluted with Tris buffer (50 mM NaCl, 25 mM Tris, 0.1% NP40) to the volume of 10 ml to dilute the concentration of SDS before immunoprecipitation. 1 mg of cell lysate was incubated with 20 μl of anti-HA affinity resin for 4 hr at 4° C. The resin was then washed three times with IP washing buffer and boiled in 30 μl of protein loading buffer for 10 min. The supernatant was then resolved by SDS-PAGE and the mono-ADP ribosylation was detected by western blot using mono-ADP-ribose binding reagent at dilution of 1:1000 (Millipore, MABE 1076).

### Generation of Inducible TIPARP Overexpression Cells

FLAG-TiPARP gene was cloned into lentiviral pUltra-dox vector (doxycycline inducible multicistronic lentiviral gene expression system). Tet-on inducible HCT116 stable cell lines were generated as previously reported (Gomez-Martinez, M. et al., J Vis Exp, e5017L (2013)) with modified procedures. Briefly, HEK 293T cells were transfected with pUltra-puro-rtTA3 (rtTA3, reverse tetracycline-controlled transactivator 3) and pUltra-dox-TIPARP plasmid to produce lentivirus. HCT116 cells were first transduced with viral particles carrying rtTA3 construct and selected with 2 μg/ml puromycin for 10 days. Ceils stably expressing rtTA3 wore further infected with virus carrying pUltra-dox-TIPARP. Expression of FLAG-TiPARP is induced by the treatment of 10 μg/ml doxycycline for 24 hr. Successful induction is confirmed by performing anti-flag immunofluorescence confocal imaging and western blot.

### Cell Proliferation Assay

HCT116 and MCF-7 cells stably expressing luciferase (control “Ctrl”) shRNA or TIPARP shRNA were seeded in 24-well plate at a density of 3,000 cells/well. 24 hr after seeding, cells in each well were washed with PBS, fixed with ice-cold methanol for 10 min and stained with 0.5% crystal violet (m/v, in 25% methanol). Stained cells were then washed and air-dried. Crystal violet stain in each well was eluted with 500 μl of 10% acetic acid in water. 100 μl of sample from each well was measured in a 96-well plate at 550 nm. From day 0 to day 5, cells were fixed every 24 hr to monitor the growth rate.

### Soft Agar Colony Formation Assay

In a 6-well plate, 2 ml 0.6% base low-melting point agarose (LMP) in complete medium supplemented with 10% FBS was added to each well, and allowed to solidify for 30 min at room temperature. For each well, 5×10³ HCT116 cells stably expressing luciferase (Ctrl) shRNA or TIPARP shRNA were resuspended in 1 ml of 0.3% agarose in 10% FBS McCoy's 5A medium, and plated on top of the 0.6% agarose base. 200 μl of fresh medium was added to the cells every 2 days. After 2-3 weeks of culture, colonies were stained with 0.1% crystal violet (m/v in 25% methanol) for 20 min at room temperature, rinsed with 50% methanol, and counted with ImageJ.

### Lactate and Glucose Measurements

Glucose and lactate levels in culture media were measured using the Glucose Assay Kit and Lactate Assay Kit (Biomedical Research Service Centre, SUNY Buffalo). Fresh media were added to a 24-well plate of subconfluent cells, and extracellular glucose/lactate concentration were measured 24 hours later and normalized to the number of cells in each well.

### Mice Study

Tumors were established via by subcutaneously injection of lentivirus-infected MCF-7 and HCT116 cells (5×10⁶ cells/animal) into the armpit of 4- to 6-week-old NOD scid gamma mice (NSG mice) (Jackson Laboratory, Bar Harbor, US). For TiPARP expression xenografts, HCT116 Tet-on TiPARP stable cell lines were treated with or without 20 μM doxycycline for 36 hr. Cells were then injected subcutaneously into mice. In control group, mice were fed with control diet; in Dox group, mice were fed with food containing doxycycline hyclate (625 mg per kg diet). Tumor volume (TV) was monitored daily and was calculated as V=½*length*width². At the termination of experiment, tumor tissues were harvested, weighted, and immunohistochemistry was further conducted. All animal experiments were carried out in accordance with the guidelines of the National Advisory Committee on Laboratory Animal Research and the Cornell University Institutional Animal Care and Use Committee.

### Immunohistochemistry

Tumors were fixed in 4% paraformaldehyde and embedded in paraffin. Sections were stained with hematoxylin and eosin (H&E) in accordance with standard procedures. Immunohistochemistry was performed using antibodies against CD31 (Abeam), HIF1α (Novus Biological s), and TiPARP (Sigma) according to manufacturer instructions.

### Statistics

Means, s.d., and s.e.m were analyzed using PRISM™ (v6.0) or MICROSOFT EXCEL™. P values were calculated based on two-tailed, unpaired Student's t-tests. Statistical significance was accepted for P values of <0.05. All experiments were performed at least two to three times.

### Example 2: TiPARP is a Direct Target Gene of HIFs

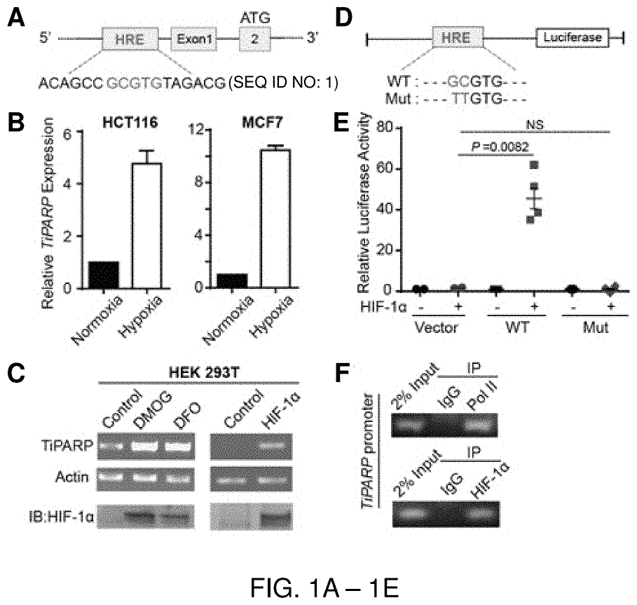

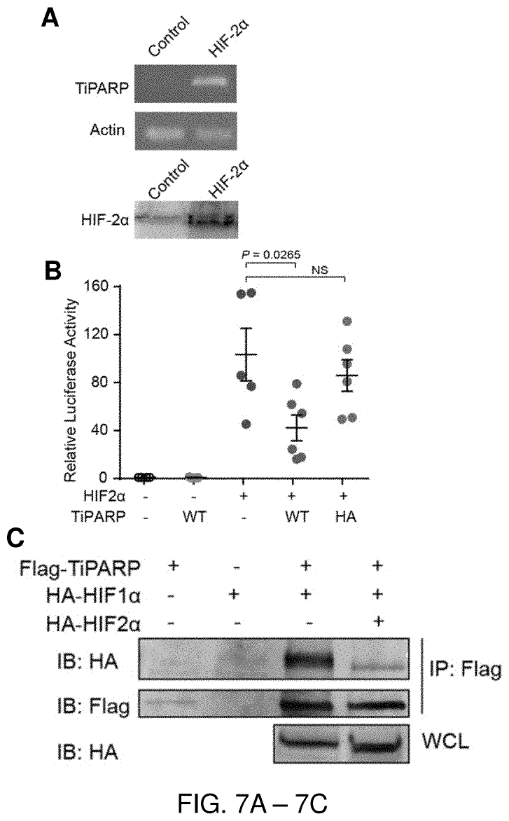

TiPARP was previously characterized as a TCDD-responsive gene regulated by AHR (Ma, Q. Arch Biochem Biophys 404, 309-316 (2002); Ma, Q. et al, Biochem Biophys Res Commun 289, 499-506, (2001)). In searching for other regulatory mechanisms for TiPARP, the inventors identified a potential hypoxia response element upstream of TiPARP exon 1 (FIG. 1A), implying that HIFs control TiPARP expression. To test this, the inventors examined mRNA level of TiPARP under hypoxia, which is a well-established model for studying HIFs function. Indeed, TiPARP mRNA was significantly up-regulated under hypoxia (<1% O₂) in both HCT116 and MCF-7 cell lines (FIG. 1B). Additionally, TiPARP mRNA level was increased by the treatment of hypoxia-mimetic agents dimethyloxalylglycine (DMOG) and desferrioxamine (DFO), or over-expression of HIF-1α (FIG. 1C) or HIF-2α (FIG. 7A).

To further confirm TiPARP is a target gene of HIFs, luciferase reporter assay was performed in HEK 293T cells with constructs with or without the regulatory region of TiPARP. Luciferase reporter constructs containing TiPARP promoter with wild-type, but not mutated hypoxia response element (HRE), displayed significantly increased luciferase activity in response to hypoxia (1% O₂) or HIF-1a over-expression (FIGS. 1D and 1E). Moreover, the binding of HIF-1α and RNA polymerase II (Pol II) to TiPARP promoter under hypoxia was validated by chromatin immunoprecipitation-polymerase chain reaction (ChIP-PCR, FIG. 1F) in 293T cells. These results demonstrate that TiPARP is a novel target gene of HIFs.

### Example 3: TiPARP Represses HIF-1 Transcriptional Activity

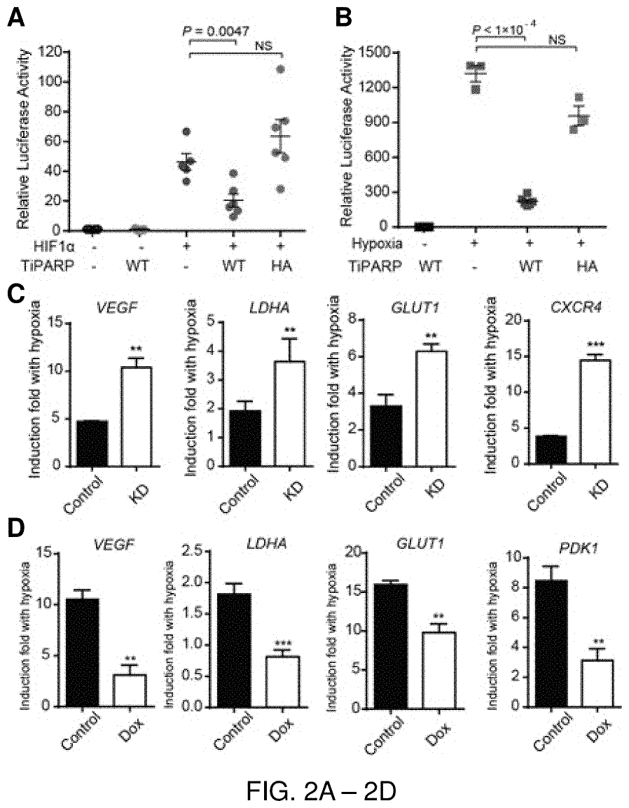

TiPARP has been documented to modulate the activity of transcription factors by ADP-ribosylation (MacPherson, L. et al. Nucleic Acids Res 41, 1604-1621, (2013); Bindesboll, C. et al, Biochem J 413, 899-910, (2016)). Thus, the inventors asked whether it also regulates HIFs transcriptional activity. Using a HRE-luciferase reporter construct, the inventors assessed whether TiPARP affects the transcriptional activity of HIF-1α. As a positive control, the reporter gene was significantly induced by HIF-1α over-expression (FIG. 2A) or hypoxia (FIG. 2B). Co-transfection of TiPARP with HIF-1α resulted in a significant decrease of reporter gene expression (FIG. 2A), indicating that HIF-1α transactivation was inhibited by TiPARP. hi addition, TiPARP dramatically repressed the transcriptional activity of endogenous HIF-1α under hypoxia (FIG. 2B). Moreover, the catalytic activity of TiPARP was required for the inhibition, as expressing catalytically inactive H532A mutant TiPARP had little effect on HIF-1α activity (FIG. 2A and FIG. 2B). Similar inhibitory effect by TiPARP was also observed on HIF-2α (FIG. 7B).

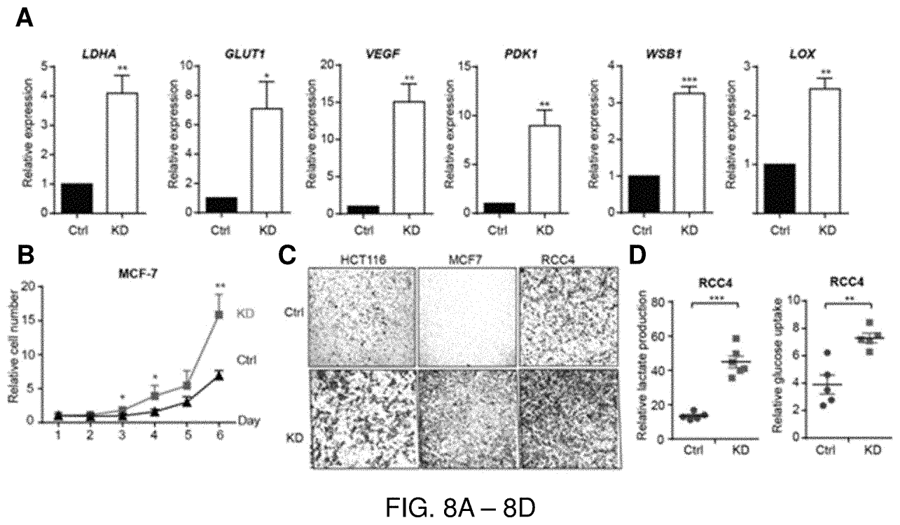

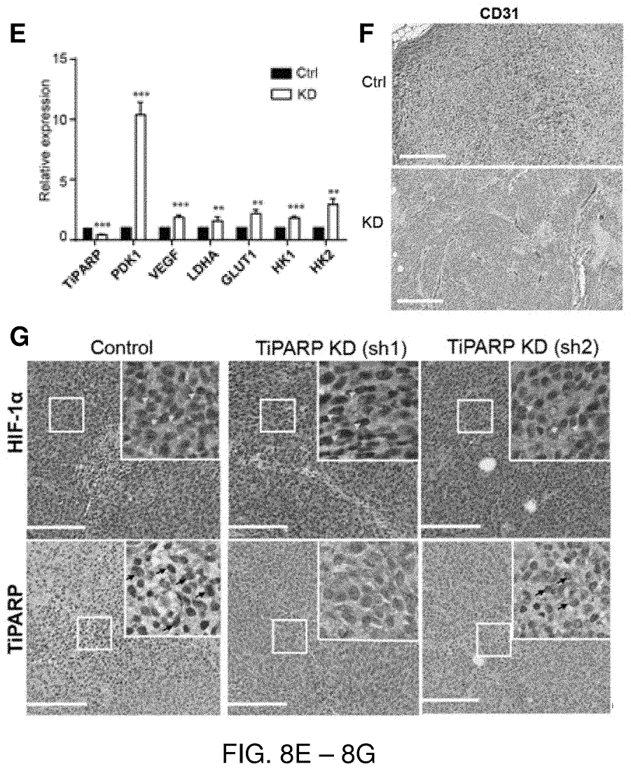

To further validate this effect in a more physiological relevant setting, the inventors examined the effect of TiPARP knockdown on HIF-1 target gene expression in HCT116 cells. The inventors found that the expression of well-characterized HIF-1 target genes, including vascular endothelial growth factor A (VEGF), glucose transporter 1 (GLUT1), lactate dehydrogenase A (LDHA), were elevated under hypoxia (FIG. 2C). The activation of these genes was significantly increased by TiPARP knockdown compared to control knockdown (FIG. 2B). The inventors also examined the mRNA level of HIF-1α target genes in a VHL-mutant clear cell renal cell carcinoma (ccRCC) cell line RCC4 that expresses stabilized HIF-1α (BaJdewijns, M. M. et al. J Pathol 221, 125-138, (2010)). The inventors observed that knocking down TiPARP promoted the expression of HIF-1α target genes in RCC4 cells (FIG. 8A). To corroborate the inhibition of TiPARP on HIF-1α, Tet-on-inducible HCT116 stable cells conditionally expressing Flag-tagged TiPARP were generated. Induction of TiPARP expression by doxycycline treatment attenuated the activation of HIF-1α target genes in response to hypoxia (FIG. 2D) Collectively, all the data above support the idea that TiPARP functions as a negative regulator of HIF-1.

### Example 4: TiPARP Interacts with and ADP-Ribosylates HIF-1α

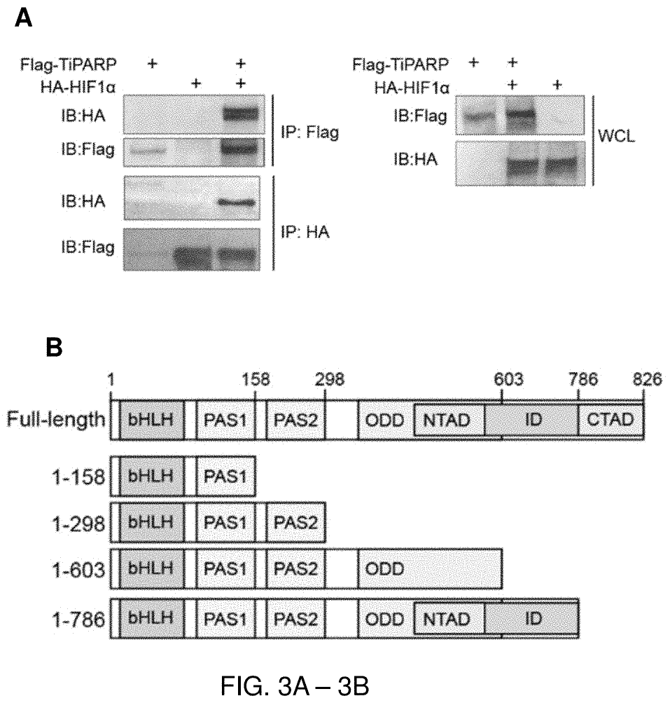

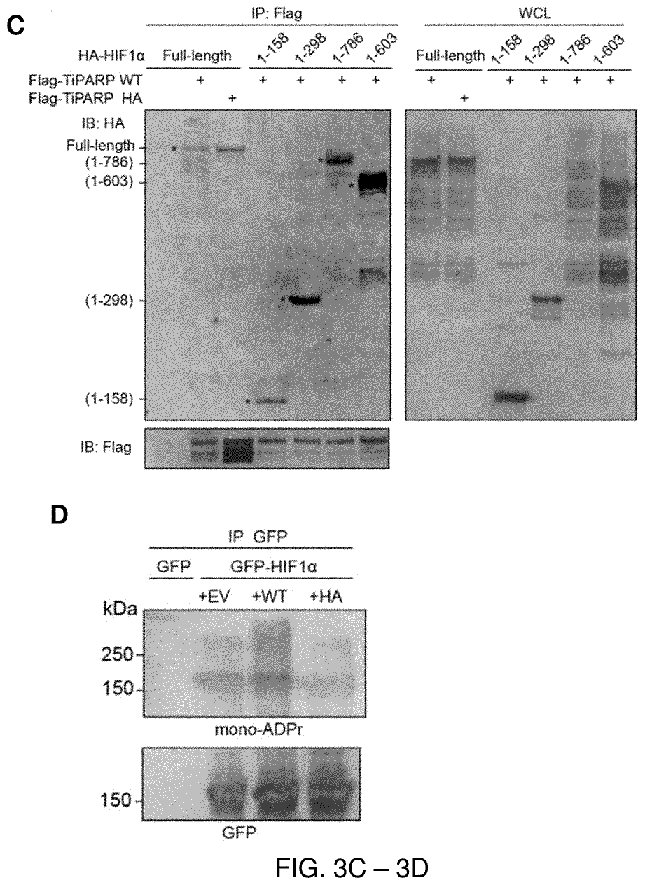

The inventors next, investigated how TiPARP regulates HIF-1. Co-immunoprecipitation experiments indicated that hemagglutinin (HA)-tagged HIF-1α (HA-HIF1α, FIG. 3A) and HA-HIF2α (FIG. 7C) interact with Flag-tagged TiPARP. Using various HIF-1α truncations for co-immunoprecipitation, the inventors found that the bHLH-PAS1 domain of HIF-1α, the conserved structure signature of bHLH-PAS family, was responsible for interacting with TiPARP (FIG. 3B).

The inventors next tested whether HIF-1α is a substrate for TiPARP. The inventors co-expressed HA-HIF-1α with wild type or inactive H532A mutant TiPARP in HEK 293T cells. The inventors then lysed cells with 4% SDS containing buffer, which denatured proteins and disrupted protein-protein interactions. HA-HIF-1α was immunoprecipitated and blotted for mono-ADP-ribosylation. The mono-ADP-ribosylation level of HIF-1α was increased by wild type TiPARP, but not the catalytic inactive H532A mutant (FIG. 3C), supporting that HIF-1α is a substrate of TiPARP.

### Example 5: TiPARP Directs HIF-1α to Nuclear Bodies and Promotes its Degradation

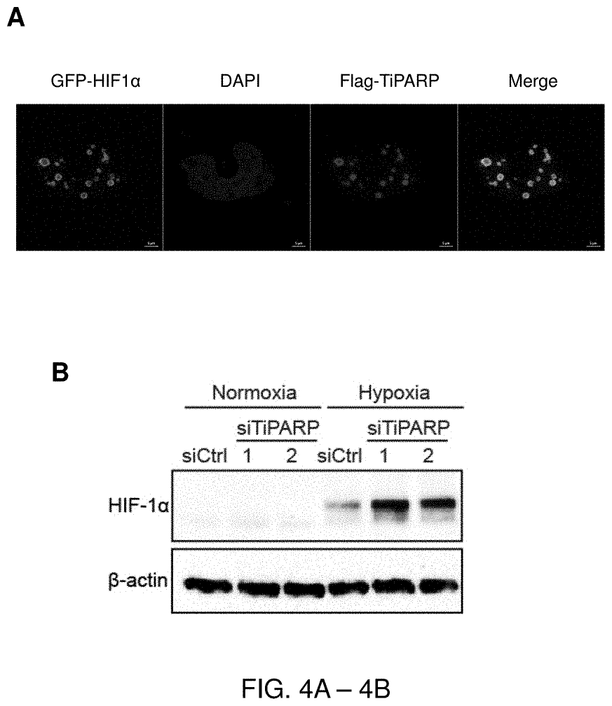

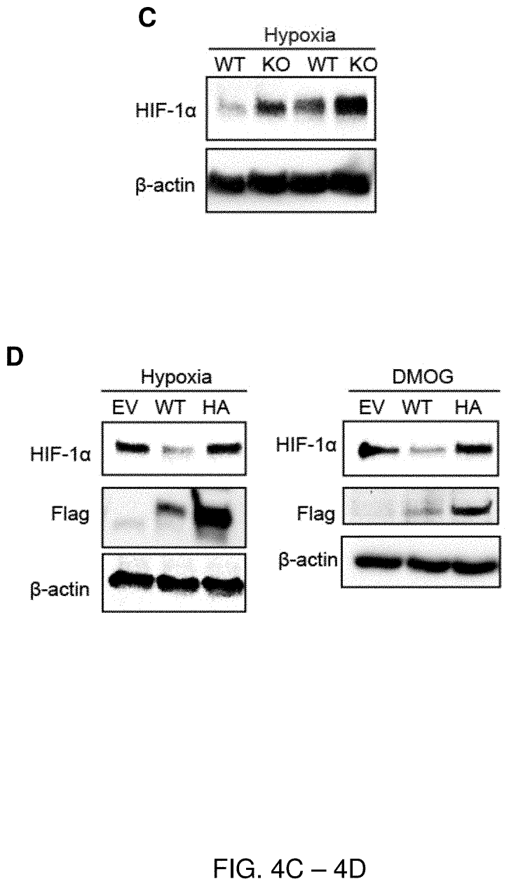

To understand how TiPARP regulates HIF-1α transcriptional activity, the inventors examined the localization of HIF-1α and TiPARP by confocal imaging. The inventors first observed that FLAG-tagged wild type TiPARP was localized in spherical subnuclear structures, whereas catalytically inactive mutant H532A lost nuclear foci accumulation (FIG. 4A).

The inventors then checked whether the protein level of HIF-1α is regulated by TiPARP. The data showed that TiPARP knockdown increased HIF-1α protein level, while overexpression decreased HIF-1α protein level (FIG. 4B-4D)

### Example 6: TiPARP Represses the Warburg Effect and Tumorigenesis

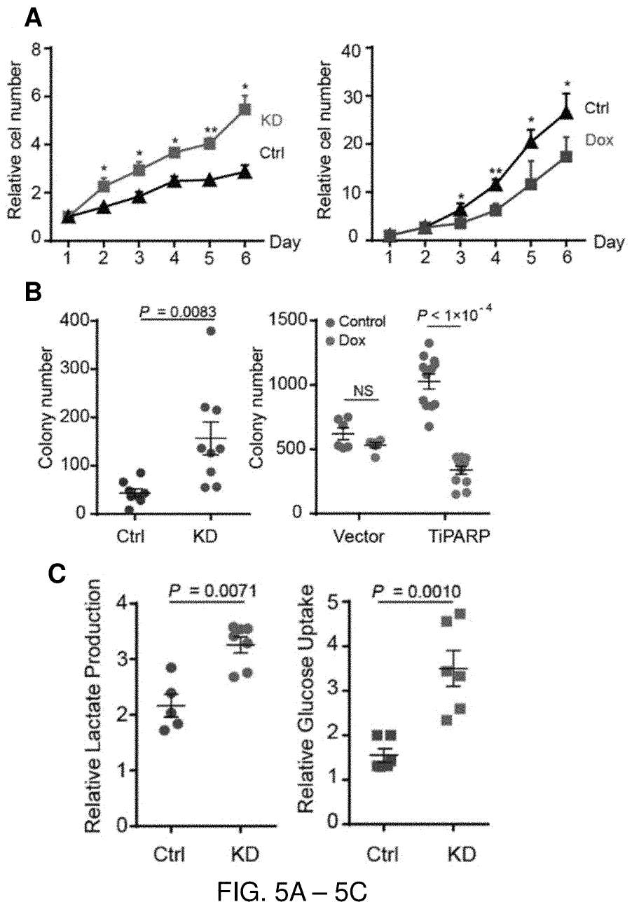

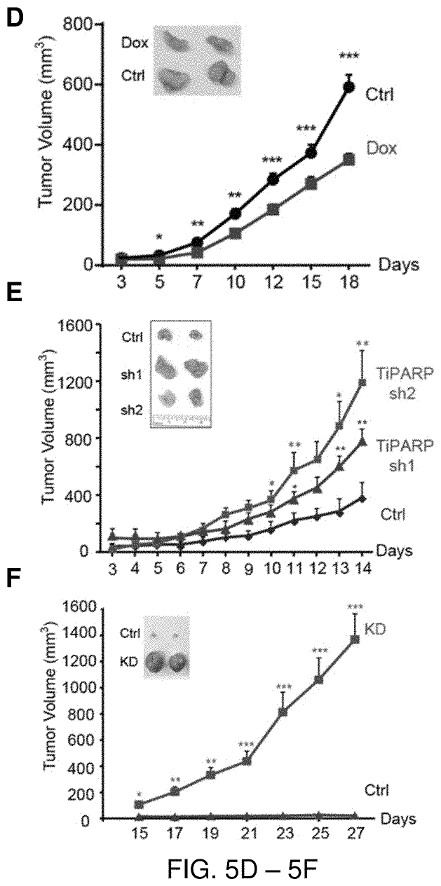

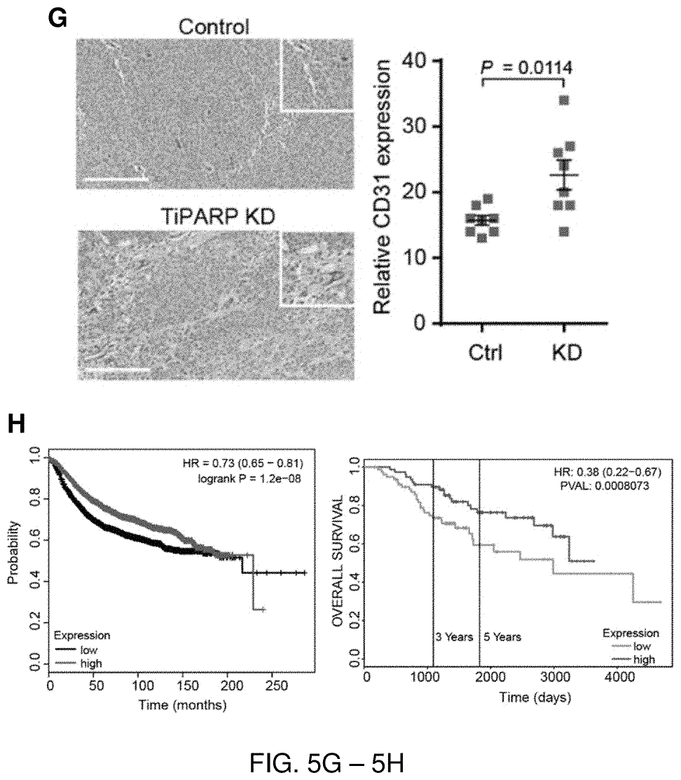

HIF-1α is overexpressed in different types of cancer (Zhong, H. et al Cancer Res 59, 5830-5835 (1999); Talks, K. I, et al Am J Pathol 157, 411-421 (2000)) and is crucial for the adaptive responses of tumors to changes in oxygenation by activating the transcription of genes involved in glucose metabolism, angiogenesis, cell survival, and invasion (Gordan, J, D. & Simon, M, C. Curr Opin Genet Dev 17, 71-77, (2007); Semenza, G. L. Nat Rev Cancer 3, 721-732, (2003); Ryan, H. E. et al. Cancer Res 60, 4010-4015 (2000); Maxwell, P. H. et al, Proc Natl Acad Sci USA 94, 8104-8109 (1997)). Elevated HIF-1α is strongly correlated with poor patient prognosis and tumor resistance to therapy (Schindl, M. et al. Clin Cancer Res 8, 1831-1837 (2002); Bachtiary, B. et al Clin Cancer Res 9, 2234-2240 (2003)). Given the inhibitory effect of TiPARP on HIF-1α, the inventors hypothesized that TiPARP may also regulate tumorigenesis and tumor growth. The inventors first examined the effect of Ti PARP on cancer cell growth by stably knocking down TiPARP or transiently overexpressing TiPARP in Tet-on-inducible HCT116 cells. Compared to control cells, TiPARP deficient cells proliferated significantly faster, while TiPARP overexpressing cells behaved in the opposite way (FIG. 5A). The cancer cell growth-promoting effect of TiPARP silencing was also observed in MCF-7 cells (FIG. 8B). Furthermore, TiPARP knockdown promoted, while TiPARP overexpression inhibited anchorage-independent growth of HCT116 cells (FIG. 5B). Collectively, this data demonstrated that TiPARP suppresses cancer cell growth. Interestingly, TiPARP depletion also enhanced cell migration in various cancer cell lines (FIG. 8C).

Cancer cells tend to shift from oxidative phosphorylation to the less energetically efficient aerobic glycolysis (the Warburg effect) to support increased requirement for biosynthesis and adapt to hypoxic microenvironment (Vander Heiden, M. G. et al., Science 324, 1029-1033, (2009)). HIF-1 mediates such metabolic reprogramming through the induction of glycolytic enzymes and glucose transporters (GLUTs) (Semenza, G. L., Curr Opin Genet Dev 20, 51-56, (2010)). To test whether TiPARP regulates cell growth by modulating metabolic shift to aerobic glycolysis, the inventors measured the lactate production and glucose uptake of cancer cells. Consistent with the data that TiPARP regulates the expression of glycolytic genes through HIF-1α, TiPARP deficient HCT116 cells consumed more glucose and released more lactate into the media than control knockdown cells in response to hypoxia (FIG. 5C). As HIF-1α is stabilized in RCC4 cells, the inventors also examined the effect of TiPARP in this cell line. Indeed, knock down of TiPARP by shRNAs led to increased glucose uptake and lactate secretion (FIG. 8D).

Next, the inventors examined whether TiPARP also suppresses tumor growth in vivo. Mice were fed with doxycycline-containing diet to induce and maintain the expression of TiPARP in Tet-on HCT116 xenografts. TiPARP overexpression resulted in smaller tumor size (FIG. 5D). Similarly, HCT116 TiPARP knockdown (KD) xenografts were larger in size than control group (FIG. 5E). The tumor-promoting effect of TiPARP knockdown was even stronger in MCF-7 xenografts (FIG. 5F). mRNA were isolated from MCF-7 tumor tissues and the expression of HIF-1α targets were quantified by qRT-PCR. As expected, glycolytic genes were significantly upregulated in TiPARP KD xenografts (FIG. BE). Since the inventors observed an increase in VEGF expression level, the inventors immunostained tumor tissues with endothelial marker CD31 to evaluate the angiogenesis in solid tumors. Compared to control group, microvessel density was higher in TiPARP KD tissues (FIG. 5G and FIG. 8F). Further, immunohistochemical staining showed that the level of HIF-1α protein negatively correlated with TiPARP; stronger nuclear staining of HIF-1α was observed in TiPARP KD tumor tissues compared to control (FIG. 8G). In addition, lower TiPARP expression strongly correlates with worse patient prognosis, according to miRpower and PROGgene analysis (Lanczky, A. et al, Breast Cancer Res Treat 160, 439-446, (2016); Goswami, C. P. & Nakshatri, P L, J Clin Bioinforma 3, 22, (2013)) (FIG. 5H). Collectively the data provide strong support that TiPARP-HIF axis is important for tumor growth in vivo and can be targeted as a potential cancer treatment strategy.

### Example 7: TiPARP Negatively Regulates the Protein Level of Oncogenic Transcription Factor HIF-1α

Once induced under hypoxia, TiPARP served as s negative regulator of HIF-1 signaling. The silencing of TiPARP increases the protein level of HIF-1α (FIG. 4A-B), while overexpressing catalytically active TiPARP significantly decreases HIF-1α protein (FIG. 4C). Collectively, the data in the present disclosure data demonstrate that TiPARP constitute a negative feedback loop for HIF-1α.

### Example 8: Compounds that Increase Expression of TiPARP in Cells

Compounds (biochanin A, diindolylmethane, resveratrol, formonometin, indole-3-carbinol, omerprazole, 4-hydroxytamoxifen, and tamoxifen) were tested and some were found to increase the transcription of TiPARP as shown in Table 1.

TABLE 1

| | TiPARP mRNA fold induction | | | | | | | |
|---|---|---|---|---|---|---|---|---|
| | Biochanin | Diindolyl- | | | indole-3- | | 4-hydroxy- | |
| | A | methane | Resveratrol | Formonometin | carbinol | Omerprazole | tamoxifen | tamoxifen |
| HCT116 | 5.6 | 3.3 | 2.1 | 1.7 | 1.5 | 0.81 | 69.2 | — |
| (colon | | | | | | | | |
| cancer) | | | | | | | | |
| SKBR3 | 8.8 | 4.2 | 3.7 | 2.5 | 1.4 | 2.3 | 84.1 | — |
| (breast | | | | | | | | |
| cancer) | | | | | | | | |
| MCF-7 | 2.5 | 8.0 | 1.1 | 2.3 | 1.4 | 1.7 | 68.9 | 14.5 |
| (breast | | | | | | | | |
| cancer) | | | | | | | | |
| BT549 | — | 3.2 | | | | | 87.8 | 56.0 |
| (breast | | | | | | | | |
| cancer) | | | | | | | | |
| A549 | | | | | | | 3.74 | 2.0 |
| (lung | | | | | | | | |
| cancer) | | | | | | | | |

IC₅₀ values of some of these compounds were also tested in various cell lines as shown in Table 2.

TABLE 2

| | IC₅₀ value (mM) | | | | |
|---|---|---|---|---|---|
| | Biochanin | resver- | Diindolyl- | 4-Hydroxy- | |
| | A | atrol | methane | tamoxifen | Tamoxifen |
| HCT116 | 20.0 | 46.0 | 39.0 | 4.0 | 8.6 |
| SKBR3 | 20.0 | 35.3 | 45.0 | 6.7 | 9.7 |
| (ER neg.) | | | | | |
| MCF-7 | 19.7 | 27.7 | 31.0 | 1.3 | 0.88 |
| (ER pos.) | | | | | |
| MDA-MB- | | 90.4 | 47.9 | 10.1 | 2.2 |
| 231 (ER | | | | | |
| negative) | | | | | |
| MDA-MB- | | 97.3 | 13.5 | 6.3 | 1.5 |
| 468 (ER | | | | | |
| negative) | | | | | |
| BT549 (ER | | 23.1 | 32.1 | 8.9 | |
| negative) | | | | | |
| HME1 | | 189 | 51.2 | 14.4 | |
| (normal | | | | | |
| mammary | | | | | |
| epithelial | | | | | |
| cell) | | | | | |
| A549 | | | | 13.6 | 26.2 |

### Example 9: Effects of TiPARP OH cMyc and ERα Protein Levels

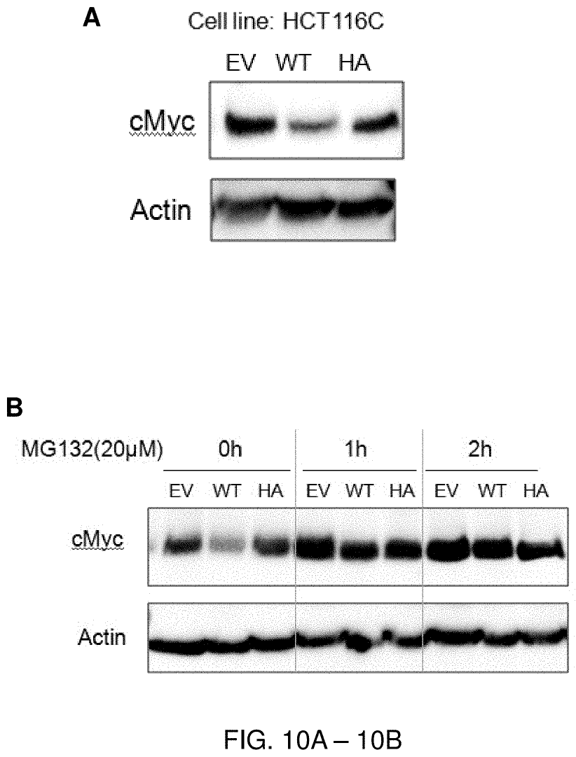

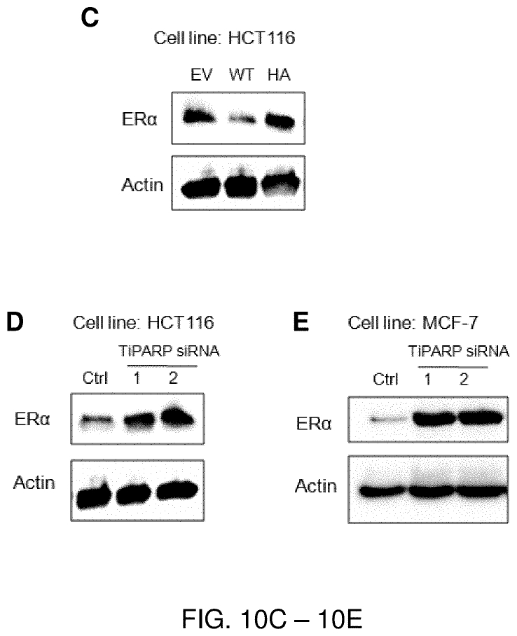

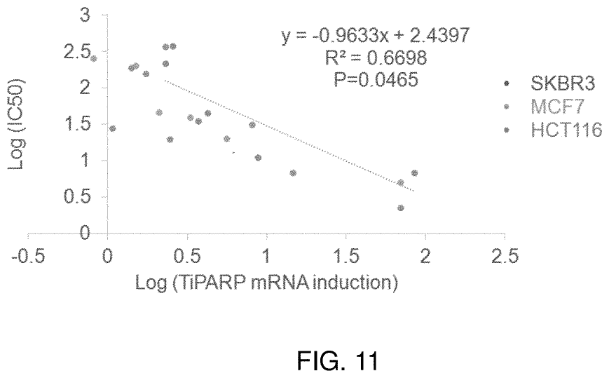

The inventors also observed that overexpression of active TiPARP decreases cMyc protein levels. Wild type (active) or inactive HA mutant of TiPARP was expressed in HCT 116C cells. WT overexpression significantly decreased cMyc levels (FIG. 10A).

The inventors also discovered the TiPARP regulates cMyc protein levels by regulating the protein degradation pathway. It was found that 20 μM MG132 (protease inhibitor) treatment effectively blocked the effect of TiPARP overexpression in cells (FIG. 10B).

The inventors also investigated the effect of TiPARP overexpression and knockdown on ERα protein levels. Overexpression of active TiPARP (WT) caused a decrease in ERα protein levels, whereas overexpression of the inactive mutant (HA) had no effect (FIG. 10C) in HCT116 cell s. Moreover, knockdown of TiPARP in HCT116 and MCF-7 cells caused ERα protein levels to increase (FIG. 10D and FIG. 10E).

### Example 10

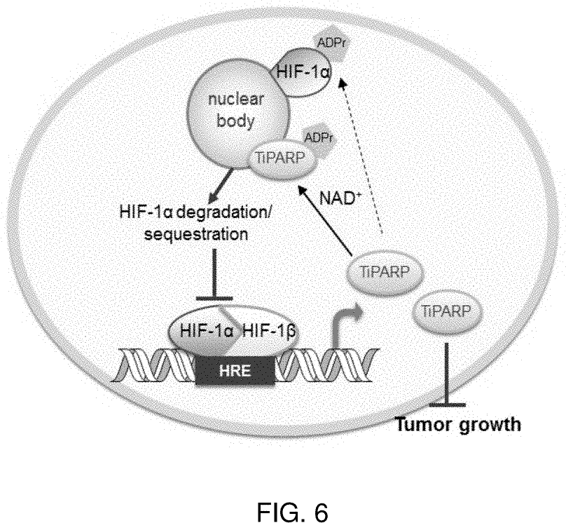

The data presented in this disclosure demonstrate that TiPARP is a target of HIF-1α and once expressed, acts as a negative regulator of hypoxic signaling (FIG. 6). TiPARP interacts with and ADP-ribosylates HIF-1α. Mono-ADP-ribosylation serves as a signal that targets substrates (including HIF-1α and auto-modified TiPARP itself) to a specific subnuclear compartment, the TiPARP nuclear bodies, which promotes HIF-1α degradation and thus suppress its transcriptional activity. Through inhibition of HIF-1 signaling, TiPARP suppresses the Warburg effect and tumorigenesis in xenograft models of human colon and breast cancer. Although ADP-ribosylation has been reported to modulate transcription (Kraus, W. L. & Lis, J. T. Cell 113, 677-683 (2003)), the work described here establishes a novel mechanism via which ADP-ribosylation could regulate transcription. To date, ADP-ribose binding domains has been found in numerous proteins, with macrodomains being the best-characterized readers for both mono- and poly-ADP-ribose (Cohen, M. S, & Chang, P. Nat Chem Biol 14, 236-243, (2018); Feijs, K. L. et. al., Nat Rev Mol Cell Biol 14, 443-451, (2013)).

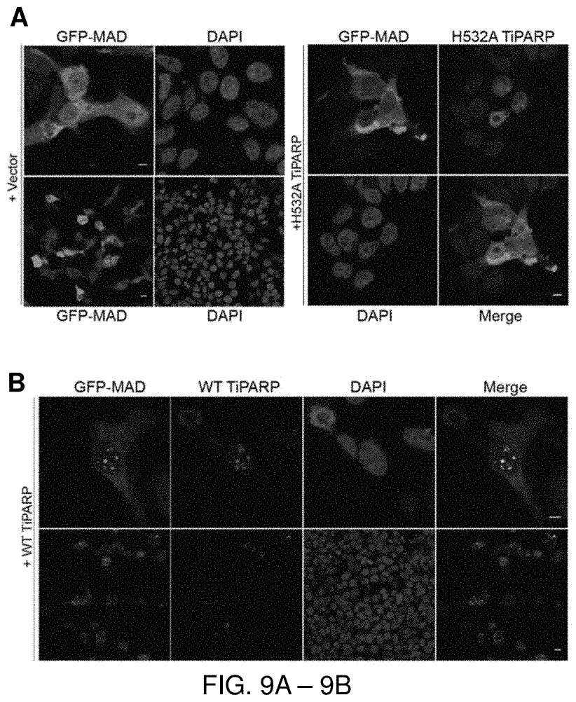

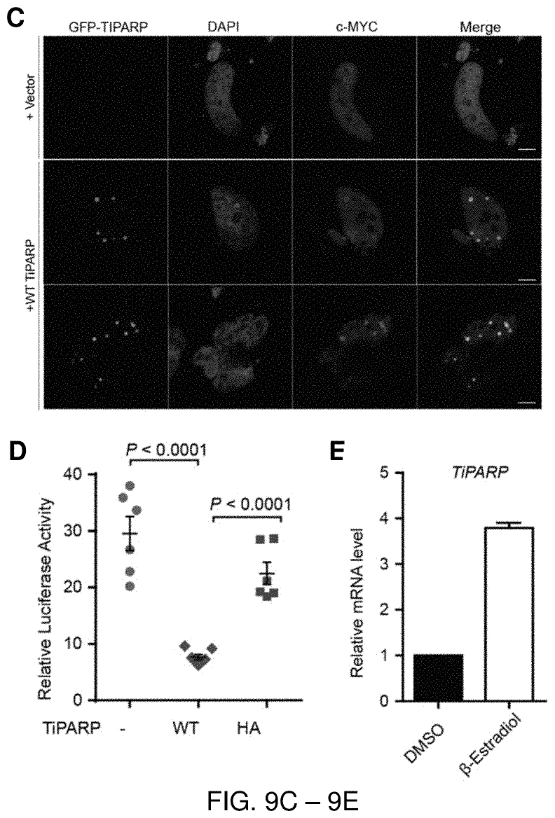

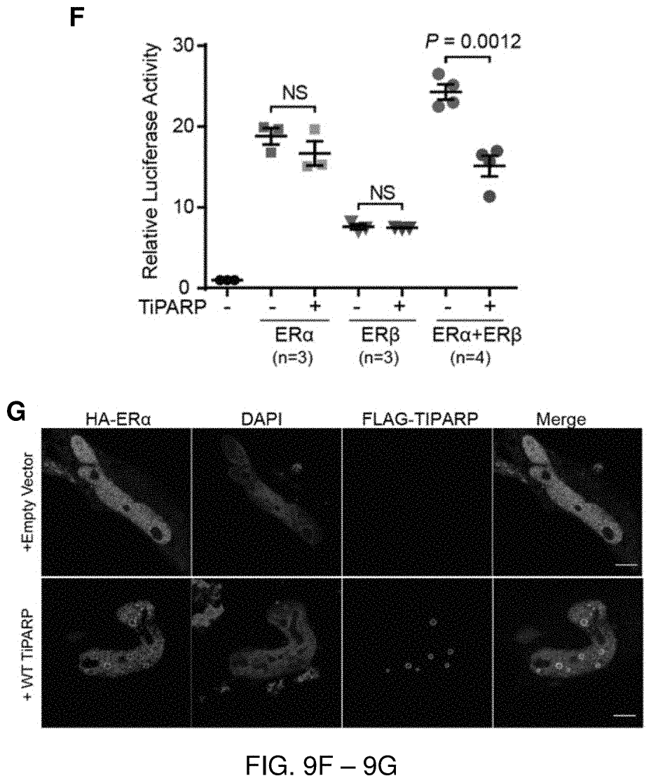

The present disclosure reveals that TiPARP-catalyzed mono-ADP-ribosylation serves as a signal for targeting transcription factors to TiPARP nuclear bodies, which leads to inhibition of their transcriptional activities. It is worth mentioning that TiPARP is maintained at low levels in cells. In response to hypoxia, dioxin, or estrogen stimulation, TiPARP expression is activated by the corresponding transcription factors. The conditional expression of TiPARP forms part of the negative feedback loop, making sure that targeting of clients to TiPARP nuclear bodies is tightly controlled. Although most of the present data focus on HIF-L some preliminary data suggest that the TiPARP-mediated regulation also applies to several other transcription factors (FIG. 9). Given the significant role of these transcription factors in oncogenic signaling, it is not surprising that these data support that TiPARP can act as tumor suppressor. The present study suggests that regulating TiPARP could be a promising alternative strategy for treating cancers.

### Example 11: The Ability of Biochanin A and 4-Hydroxy Tamoxifen to Inhibit Cancer Cell Growth is Partially TiPARP-Dependent

IC₅₀ values for Biochanin A was measured in HCT116, SKBR3 and MCF-7 cell lines where TiPARP was knocked down. The results show that TiPARP knockdown significantly increases the concentration of drug necessary to inhibit 50% growth of the cells (IC₅₀), as shown in Table 3.

TABLE 3

| | | | TiPARP KD |
|---|---|---|---|
| | | Ctrl | (40%) |
| HCT116 | IC50 (μM) | 30.0 | 54.8 |
| SKBR3 | IC50 (μM) | 52.3 | 83.3 |
| MCF-7 | IC50 (μM) | 93.4 | 173.5 |

IC₅₀ values for 4-hydroxy tamoxifen was measured in SKBR3 cell line where TiPARP was knocked down. The results show that TiPARP knockdown significantly increases the concentration of 4-hydroxy tamoxifen necessary to inhibit 50% growth of the ceils (IC₅₀), as shown in Table 4.

TABLE 4

| | | | TiPARP KD |
|---|---|---|---|
| | | Ctrl | (40%) |
| SKBR3 | IC50 (μM) | 4.9 | 10.51 |

What is claimed is:

1. A method of treating cancer in a subject in need thereof, the method comprising administering to the subject a therapeutically effective amount of a TiPARP agonist.

2. The method of claim 1, wherein the TiPARP agonist is an aryl hydrocarbon receptor (AHR) agonist or an estrogen receptor (ER) agonist.

3. The method of claim 1, wherein the TiPARP agonist interacts with TiPARP directly.

4. The method of claim 1, wherein the TiPARP agonist is a tamoxifen compound within the following generic formula: 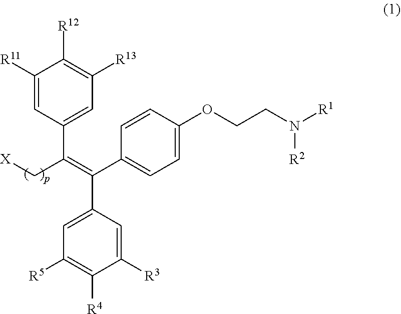
wherein:
R¹ and R² are independently selected from alkyl groups containing one to three carbon atoms, or alternatively, R¹ and R² may interconnect to form a five-membered or six-membered heterocycloalkyl ring;
R³, R⁴, and R⁵ are independently selected from hydrogen atom, halogen atom, methyl, ethyl, hydroxy (OH), methoxy (—OCH₃), and ethoxy (—OCH₂CH₃);
R¹¹, R¹², and R^{1J} are independently selected from hydrogen atom, hydroxy, and methoxy groups;
X is a hydrogen atom or halogen atom; and
p is 2 or 3.

5. The method of claim 4, wherein the tamoxifen compound has the following structure:

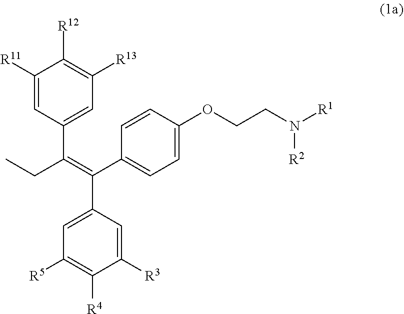

6. The method of claim 5, wherein R¹ and R² are independently selected from alkyl groups containing one to three carbon atoms.

7. The method of claim 6, wherein R¹ and are methyl groups, which corresponds to the following structure:

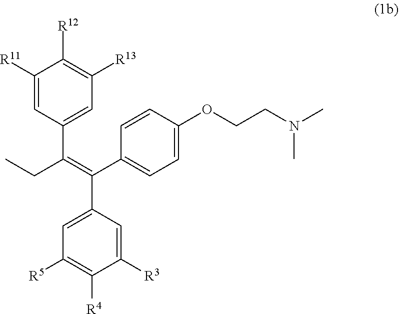

8. The method of claim 7, wherein R³ and R⁵ are hydrogen atoms and R⁴ is selected from the group consisting of halogen atom, methyl, ethyl, hydroxy (OH), methoxy (—OCH₃), and ethoxy (—OCH₂CH₃).

9. The method of claim 7, wherein R³, R⁴, and R⁵ are hydrogen atoms, which corresponds to the following structure:

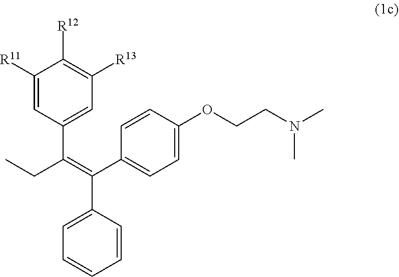

10. The method of claim 9, wherein at least one of R¹¹, R¹², and R¹³ is a hydroxy or methoxy group.

11. The method of claim 10, wherein R¹¹ and R¹² are hydrogen atoms and R¹² is a hydroxy or methoxy group.

12. The method of claim 11, wherein the compound has the following structure:

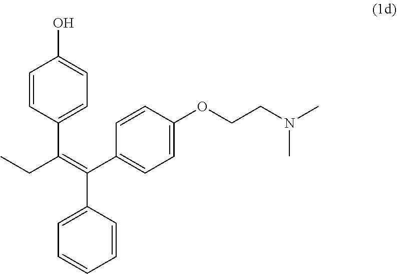 [SMILES: CC/C(=C(\c1ccccc1)c1ccc(OCCN(C)C)cc1)c1ccc(O)cc1]

13. The method of claim 1, wherein the TiPARP agonist is a flavone or isoflavone compound within the following generic formula: 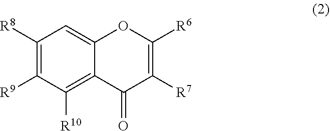
wherein:
R⁶ and R⁷ are independently selected from (i) hydrogen atom and (ii) phenyl ring optionally substituted with one or two OH and/or OCH₃ groups, provided that one of R⁶ and R⁷ is (ii);
R⁸, R⁹, and R¹⁰ are independently selected from hydrogen atom, methyl, phenyl, hydroxy, and methoxy groups, wherein said phenyl is optionally substituted with a hydroxy or methoxy group;
wherein R⁸ and R⁹ may optionally interconnect as a benzene ring, or R⁹ and R¹⁰ may optionally interconnect as a benzene ring.

14. The method of claim 13, wherein at least one of R⁸, R⁹, and R¹⁰ is a hydroxy group and none of R⁸, R⁹, and R¹⁰ interconnect.

15. The method of claim 14, wherein at least two of R⁸, R⁹, and R¹⁰ are hydroxy groups.

16. The method of claim 15, wherein one of R⁶ and R⁷ is a phenyl ring substituted with an OH or OCH₃ group.

17. The method of claim 16, wherein the TiPARP agonist has the following structure:

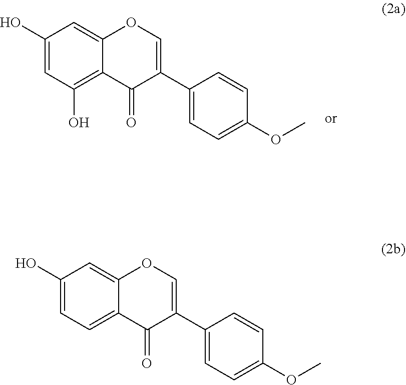 [SMILES: COc1ccc(-c2coc3cc(O)cc(O)c3c2=O)cc1 ; COc1ccc(-c2coc3cc(O)ccc3c2=O)cc1]

18. The method of claim 13, wherein R⁹ and R¹⁰ interconnect as a benzene ring and R⁸ is a hydrogen atom, which corresponds to the following structure:

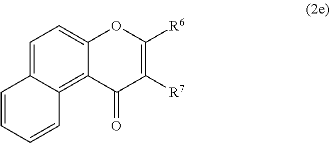

19. The method of claim 18, wherein one of R⁶ and R⁷ is an unsubstituted phenyl ring.

20. The method of claim 19, wherein the compound has the following structure:

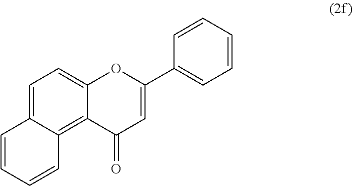 [SMILES: O=c1cc(-c2ccccc2)oc2ccc3ccccc3c12]

21. The method of claim 1, wherein the TiPARP agonist is an indolyl-containing compound.

22. The method of claim 21, wherein the indolyl-containing compound is a diindolylmethane compound having the following structure:

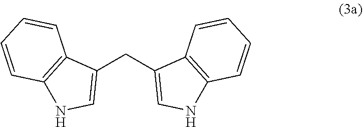 [SMILES: c1ccc2c(Cc3c[nH]c4ccccc34)c[nH]c2c1]

23. The method of claim 21, wherein the indolyl-containing compound is an indole-3-carbinol compound having the following structure:

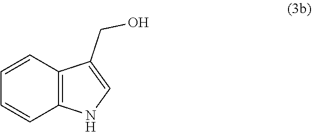 [SMILES: OCc1c[nH]c2ccccc12]

24. The method of claim 1, wherein the TiPARP agonist is a hydroxylated or methoxylated stilbene compound.

25. The method of claim 24, wherein the stilbene compound is resveratrol, which has the following structure:

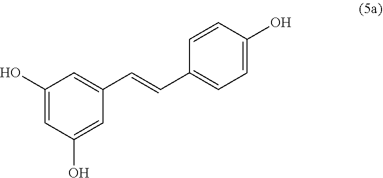 [SMILES: Oc1ccc(/C=C/c2cc(O)cc(O)c2)cc1]

26. The method of claim 1, wherein the TiPARP agonist is a chlorinated dibenzo-p-dioxin (CDBD) compound within the following generic formula: 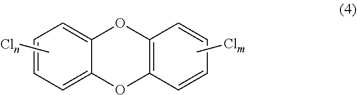
wherein n represents a number between 0 and 4, and wherein m represents a number between 0 and 4, provided that the sum of m and n is at least 1.

27. The method of claim 26, wherein the CDBD compound is tetrachlorodibenzo-p-dioxin (TCDD), which corresponds to the following structure:

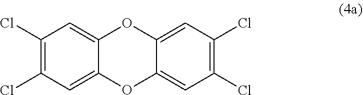 [SMILES: Clc1cc2c(cc1Cl)Oc1cc(Cl)c(Cl)cc1O2]

28. The method of claim 1, wherein the TIPARP agonist is an agent that leads to elevated expression of the TiPARP protein.

29. The method of claim 28, wherein the TIPARP agonist is an expression vector encoding an exogenous TiPARP protein.

30. The method of claim 29, wherein the expression of the exogenous TiPARP is inducible.

31. The method of claim 1, wherein the cancer is associated with elevated expression of HIT-1α.

32. The method of claim 1, wherein the cancer is selected from the group consisting of breast cancer, colon cancer, lung cancer, skin cancer, brain cancer, blood cancer, cervical cancer, liver cancer, prostate carcinoma, pancreas carcinoma, gastric carcinoma, ovarian carcinoma, renal cell carcinoma, mesothelioma, and melanoma.

33. The method of claim 4, wherein the cancer is not breast cancer.

34. The method of claim 33, wherein the cancer is lung or colon cancer.


SEQUENCE LISTING (39 sequences)

SEQ ID NO: 1 - DNA, 17 bp, Artificial Sequence
  oligonucleotide
acagccgcgtgtagacg

SEQ ID NO: 2 - DNA, 45 bp, Artificial Sequence
  oligonucleotide
agtcaggaattctatggaaatggaaaccaccgaacctgagccaga

SEQ ID NO: 3 - DNA, 54 bp, Artificial Sequence
  oligonucleotide
agtcagggatccctacttatcgtcgtcatccttgtaatccaggatatcatttgc

SEQ ID NO: 4 - DNA, 42 bp, Artificial Sequence
  oligonucleotide
aaatgagagacatttatttgctggaacatcccaggatgtggt

SEQ ID NO: 5 - DNA, 47 bp, Artificial Sequence
  oligonucleotide
aaataaatgtctctcatttattatcctgtcacggccaaacattttcc

SEQ ID NO: 6 - DNA, 52 bp, Artificial Sequence
  oligonucleotide
agtcagctcgagagatcttgtctcaataagattttaaatgtaaagattttca

SEQ ID NO: 7 - DNA, 33 bp, Artificial Sequence
  oligonucleotide
agtcagaagcttgtgcggtggacttatgctccc

SEQ ID NO: 8 - DNA, 33 bp, Artificial Sequence
  oligonucleotide
tccttcctcacagccttgtgtagacgcggaccc

SEQ ID NO: 9 - DNA, 33 bp, Artificial Sequence
  oligonucleotide
ggctgtgaggaaggaaggcgcgtgccgcgtggg

SEQ ID NO: 10 - DNA, 29 bp, Artificial Sequence
  oligonucleotide
tatttgcgtgtgaggtaacttctggatgc

SEQ ID NO: 11 - DNA, 25 bp, Artificial Sequence
  oligonucleotide
tcacacgcaaatagctgatggtaag

SEQ ID NO: 12 - DNA, 33 bp, Artificial Sequence
  oligonucleotide
gccttgtgaaaaagggttaagaacaaaacacac

SEQ ID NO: 13 - DNA, 34 bp, Artificial Sequence
  oligonucleotide
ttcttaaccctttttcacaaggccatttctgtgt

SEQ ID NO: 14 - DNA, 34 bp, Artificial Sequence
  oligonucleotide
atgatatgtttactaaaggataagtcaccacagg

SEQ ID NO: 15 - DNA, 36 bp, Artificial Sequence
  oligonucleotide
gacttatcctttagtaaacatatcatgatgagtttt

SEQ ID NO: 16 - DNA, 33 bp, Artificial Sequence
  oligonucleotide
gacttccagttacgttaattcgatcagttgtca

SEQ ID NO: 17 - DNA, 33 bp, Artificial Sequence
  oligonucleotide
gaattaacgtaactggaagtcatcatccattgg

SEQ ID NO: 18 - DNA, 35 bp, Artificial Sequence
  oligonucleotide
gttacagtattccagtagactcaaatacaagaacc

SEQ ID NO: 19 - DNA, 32 bp, Artificial Sequence
  oligonucleotide
agtctactggaatactgtaactgtgctttgag

SEQ ID NO: 20 - DNA, 30 bp, Artificial Sequence
  oligonucleotide
tagactgctggggcaataaatggatgaaag

SEQ ID NO: 21 - DNA, 32 bp, Artificial Sequence
  oligonucleotide
catttattgccccagcagtctacatgctaaat

SEQ ID NO: 22 - DNA, 58 bp, Artificial Sequence
  oligonucleotide
ccggaggtctttgaggccaatattactcgagtaatattggcctcaaagaccttttttg

SEQ ID NO: 23 - DNA, 58 bp, Artificial Sequence
  oligonucleotide
ccgggaaggcaagctactctcataactcgagttatgagagtagcttgccttctttttg

SEQ ID NO: 24 - DNA, 20 bp, Artificial Sequence
  oligonucleotide
ctacctccaccatgccaagt

SEQ ID NO: 25 - DNA, 20 bp, Artificial Sequence
  oligonucleotide
gcagtagctgcgctgataga

SEQ ID NO: 26 - DNA, 20 bp, Artificial Sequence
  oligonucleotide
cgggccaagagtgtgctaaa

SEQ ID NO: 27 - DNA, 20 bp, Artificial Sequence
  oligonucleotide
tgacgataccggagccaatg

SEQ ID NO: 28 - DNA, 20 bp, Artificial Sequence
  oligonucleotide
accaggacagccaatacaag

SEQ ID NO: 29 - DNA, 20 bp, Artificial Sequence
  oligonucleotide
cctcggtcactcatcttcac

SEQ ID NO: 30 - DNA, 27 bp, Artificial Sequence
  oligonucleotide
cgtactataggtgaacttttagctcct

SEQ ID NO: 31 - DNA, 23 bp, Artificial Sequence
  oligonucleotide
ccaaaggaaaactgctttactgg

SEQ ID NO: 32 - DNA, 20 bp, Artificial Sequence
  oligonucleotide
ctcctgtgcaaaatgccaac

SEQ ID NO: 33 - DNA, 21 bp, Artificial Sequence
  oligonucleotide
cctagagctcactagtcacag

SEQ ID NO: 34 - DNA, 21 bp, Artificial Sequence
  oligonucleotide
tgggtggttgtgttccagttt

SEQ ID NO: 35 - DNA, 22 bp, Artificial Sequence
  oligonucleotide
atgcaatagcaggacaggatga

SEQ ID NO: 36 - DNA, 20 bp, Artificial Sequence
  oligonucleotide
ggcagatttgaatgccatga

SEQ ID NO: 37 - DNA, 21 bp, Artificial Sequence
  oligonucleotide
tggacagcctccgtagttggt

SEQ ID NO: 38 - DNA, 20 bp, Artificial Sequence
  oligonucleotide
gatcattgctcctcctgagc

SEQ ID NO: 39 - DNA, 20 bp, Artificial Sequence
  oligonucleotide
actcctgcttgctgatccac